(19) United States
(12) Patent Application Publication
(10) Pub. No.: US 2026/0125757 A1
(43) Pub. Date: May 7, 2026

(54) Methods for Diagnosing Infectious Disease and Determining HLA Status Using Immune Repertoire Sequencing

(71) Applicants: Adaptive Biotechnologies Corporation, Seattle, WA (US); Fred Hutchinson Cancer Center, Seattle, WA (US)

(72) Inventors: Ryan O. Emerson, Seattle, WA (US); Harlan S. Robins, Seattle, WA (US); Mark J. Rieder, Seattle, WA (US); William S. Dewitt, III, Seattle, WA (US); Christopher S. Carlson, Seattle, WA (US)

(21) Appl. No.: 19/321,001

(22) Filed: Sep. 5, 2025

Related U.S. Application Data
(63) Continuation of application No. 17/343,552, filed on Jun. 9, 2021, now Pat. No. 12,428,682.
Application No. 17/343,552 is a division of application No. 15/553,040, now Pat. No. 11,047,008.
(60) Provisional application No. 62/120,249, filed on Feb. 24, 2015.
(60) Provisional application No. 62/157,249, filed on May 5, 2015.
(60) Provisional application No. 62/215,630, filed on Sep. 8, 2015.

(51) Int. Cl. C12Q 1/6881; C12Q 1/68; C12Q 1/6869; C12Q 1/6883; C12Q 1/70

(52) U.S. Cl. CPC C12Q 1/6881; C12Q 1/68; C12Q 1/6883; C12Q 1/70; C12Q 1/6869; C12Q 2600/158; C12Q 2600/172

(57) ABSTRACT

Methods are provided for predicting a subject's infection status using high-throughput T cell receptor sequencing to match the subject's TCR repertoire to a known set of disease-associated T cell receptor sequences. The methods of the present invention may be used to predict the status of several infectious agents in a single sample from a subject. Methods are also provided for predicting a subject's HLA status using high-throughput immune receptor sequencing.

### CROSS REFERENCE TO RELATED APPLICATIONS

This application is a continuation of U.S. patent application Ser. No. 17/343,552, filed on Jun. 9, 2021, which is a divisional of U.S. patent application Ser. No. 15/553,040, filed on Aug. 23, 2017, now U.S. Pat. No. 11,047,008, which is a national stage filing under 35 U.S.C. 371 of International Patent Application No. PCT/US2016/019343, filed on Feb. 24, 2016, which claims the benefit of U.S. Provisional Patent Application No. 62/120,249, filed on Feb. 24, 2015, U.S. Provisional Patent Application No. 62/157,249, filed on May 5, 2015, and U.S. Provisional Patent Application No. 62/215,630, filed on Sep. 8, 2015. The contents of each of these applications are herein incorporated by reference in their entirety.

### REFERENCE TO AN ELECTRONIC SEQUENCE LISTING

The contents of the electronic sequence listing (ADBS-024CON_SEQLIST; Size: 141,920 bytes; and Date of Creation: Sep. 5, 2025) is herein incorporated by reference in its entirety.

### BACKGROUND OF THE INVENTION

The cellular adaptive immune system conveys broad protection against infection by pathogens through the development of a vast and highly diverse repertoire of T cell receptor (TCR) genes, which encode cell-surface T cell receptors with randomized antigen specificity. The ability of a subject's adaptive immune system to adequately address an incipient infection relies on activation of an appropriate antigen-specific T-cell receptor (TCR). TCR-antigen interaction is mediated by the cell-surface presentation of foreign peptides by pathogen-infected cells in the context of major histocompatibility complex (MHC) proteins. Specifically, CD8⁺ T cells recognize antigen in the context of MHC class I proteins. Since MHC class I proteins are encoded by the human leukocyte antigen (HLA) loci A, B, and C, which are highly polymorphic, the antigen specificity of a TCR is modulated across individuals by HLA context.

When an antigen has been encountered, activated T cells proliferate by clonal expansion and reside in the memory T cell compartment for many years as a clonal population of cells (clones) with identical-by-descent rearranged TCR genes (Arstila T P, et al. A direct estimate of the human alphabeta T cell receptor diversity. Science 286:958-961, 1999). Protection against future exposure to disease-causing pathogens is conferred by the ability of activated T cells to form long-lasting memory responses.

The majority of TCR diversity resides in the ß chain of the TCR alpha/beta heterodimer. Each T cell clone is encoded by a single TCRß allele that has been randomly rearranged from the germ-line TCRß locus to form a mature TCRß gene. Immense diversity is generated by combining noncontiguous TCRß variable (V), diversity (D), and joining (J) region gene segments, which collectively encode the CDR3 region, the primary region of the TCRß locus for determining antigen specificity. Deletion and template-independent insertion of nucleotides during rearrangement at the Vβ-Dβ and Dβ-Jβ junctions further add to the potential diversity of receptors that can be encoded (Cabaniols J P, et al. Most alpha/beta T cell receptor diversity is due to terminal deoxynucleotidyl transferase. J Exp Med 194:1385-1390, 2001). Typically, at a given point in time, a healthy adult expresses approximately 10 million unique TCRß chains on their 10¹² circulating T cells (Robins H S, et al. (2009) Comprehensive assessment of T-cell receptor beta-chain diversity in alphabeta T cells. Blood 114:4099-4107). However, observing the same TCRß chain independently in two individuals is thousands of times more common than would be expected if all rearrangements were equally likely (Robins H S, et al. (2010) Overlap and effective size of the human CD8+ T cell receptor repertoire. Science Translational Medicine 2: 47ra64). It is expected that there are many TCRβ sequences (especially those with few or no insertions) that are present in the naïve repertoires of most individuals and that these TCRβ sequences will reliably proliferate upon exposure to their associated antigen (V. Venturi, et al., The molecular basis for public T-cell responses? Nature reviews. Immunology 8, 231-238 (2008); published online EpubMar (10.1038/nri2260)). This over-representation of specific TCRß sequence rearrangements in the naïve T cell repertoire forms the basis for public T cell responses.

Public T-cell responses occur when T cells bearing identical T-cell receptors (TCRs) are observed to dominate the response to the same antigenic epitope in multiple individuals. Many pathogenic antigens are known to induce such a public T cell response, in which a pathogenic antigen is targeted by the same T cell receptor sequence (and found to be immunodominant) in multiple individuals with specific HLA isotypes. H. Li et al., Determinants of public T cell responses. Cell research 22, 33-42 (2012); published online EpubJan (10.1038/cr.2012.1); H. Li, et al., Recombinatorial biases and convergent recombination determine inter-individual TCRbeta sharing in murine thymocytes. Journal of immunology (Baltimore, Md.: 1950) 189, 2404-2413 (2012); published online EpubSep 1 (10.4049/jimmunol.1102087). In other words, public T cell responses are observed when the space of potential high-avidity TCRß chains that could bind to a particular antigen-MHC complex includes one or more TCRß chains that also have a high likelihood of existing in the naïve repertoire at any given time. Thus, sequences associated with a public T cell response will only be intermittently present in the naïve compartment of subjects that have not been exposed to a particular antigen; however, such clones should consistently appear in the T cell repertoire of subjects who have been exposed to the antigen, having undergone clonal expansion after antigen stimulation.

Previous work on public T cell responses has identified individual examples of public T cell responses to diseases (including CMV, EBV, influenza, multiple sclerosis and other disease malignancies and autoimmune conditions), (Venturi V, et al. (2008) J Immunol 181:7853-7862). These studies were limited by sequencing depth and the size of investigational cohorts. Typically, these public T cell responses were studied in the context of single antigens in a single HLA context, usually using antigen-MHC tetramers to purify antigen-specific T cells. However, because of technical limitations, only a relatively small number of public T cell responses have been identified to date. Not surprisingly, these results have been limited to the high frequency, easily observable public T cell responses that dominate the immune response to their target antigens. Therefore, these public responses represent only TCRß sequences that are common to the naïve T cell compartment of nearly all adult subjects (i.e., available for antigen response), and misses both rare T cell rearrangements which are present in only some antigen-experienced patients due to rarity among naïve T cell sequences and T cell responses that are not immunodominant and therefore never comprise a sufficient fraction of the total T cell repertoire to be reliably observed with limited sequencing depth.

Further, TCR-antigen interaction is mediated by the cell-surface presentation of foreign peptides by pathogen-infected cells in the context of major histocompatibility complex (MHC) class I proteins. Since MHC class I proteins are encoded by the human leukocyte antigen (HLA) loci A, B, and C, which are highly polymorphic, the antigen specificity of a TCR is modulated across individuals by HLA context.

The binding of T cell receptors to antigens is mediated by MHC proteins, which present antigen on the surface of cells. MHC are encoded in humans by the HLA loci, which are highly polymorphic. This polymorphism leads to heterogeneous T cell responses to the same antigen across individuals and to differential positive and negative selection of specific T cell receptor sequences during thymic training. Determination of an individual's HLA alleles (HLA type) has several clinical applications. One example of a clinical application for HLA typing is testing an individual's suitability as a bone marrow transplant donor.

There is a need for improved methods to diagnose and/or predict an individual's status for an infectious disease, such as CMV, EBV, HPV, small pox, and others, with increased sensitivity and accuracy. Diagnostic methods are needed that harness the information about an individual's T cell receptor sequence profile, including the presence of public T cell clones, and assess the individual's infectious disease status based on the T cell receptor sequence profile. There is also a need for inferring an individual's HLA type based on the individual's T cell receptor sequence profile. The present invention fulfills these needs and provides further related advantages.

### SUMMARY OF THE INVENTION

The present disclosure is based, in part, on methods of determining or predicting the presence or absence of one or more infectious disease agents in a subject of known or unknown infection status through immunological quantification techniques combined with mathematical modeling.

The present disclosure provides a method of predicting the presence or absence of an infection in a subject of unknown infection status. In one embodiment, the genomic DNA of a sample comprising T cells obtained from the subject is subjected to amplification and high throughput sequencing to determine a T cell receptor (TCR) profile comprising unique TCR complementarity determining region 3 (CDR3) amino acid sequences. In a particular embodiment, the TCR profile is then compared against a database of previously identified diagnostic public T cell receptor sequences that are known to be statistically significantly associated with the infection. In one embodiment, a first score for the subject is then generated by determining the proportion of unique TCR sequences in the profile of the subject that match the public TCR sequences in the database. In this embodiment, the first score is input into an algorithm that compares the first score of the subject with the known infection statuses of a plurality of subjects of known infection status. In a further embodiment, an estimated probability of infection status is then determined for the subject as the algorithm output. In some embodiments, the method further comprises an initial step of obtaining a sample comprising T cells from the subject.

In particular embodiments, the present disclosure additionally provides a method of predicting the presence or absence of one or more viral infections in a subject of unknown infection status. In a further embodiment, the method comprises determining a profile of unique TCR sequences from a sample obtained from the subject (e.g., a sample comprising T cells), and inputting these unique TCR sequences into one or more algorithms. In one embodiment, the one or more algorithms are generated by determining at least 100,000 unique TCR sequences from each of a plurality of subjects of known infection status for each of the one or more infections. In some embodiments, the method further comprises an initial step of obtaining a sample from the subject. In some embodiments, unique TCR sequences are statistically identified that correlate with the presence or absence of each of the one or more infections, thus generating a score which is predictive of the presence or absence of each of the one or more infections. In these embodiments, the scores are then input into a logistic regression model trained on each of the plurality of subjects of known infection status for each of the one or more infections, the output of which is the prediction as to whether the subject is either positive or negative for each of the one or more infections.

In some embodiments the one or more infections are from a cytomegalovirus (CMV), an Epstein-Barr virus (EBV), a Herpes simplex virus (HSV) or a small pox virus.

In particular embodiments, the present disclosure also provides a method for predicting the presence or absence of a CMV infection in a subject of unknown infection status. In one embodiment, the genomic DNA of a sample comprising T cells obtained from a subject is subjected to amplification and high throughput sequencing to determine a TCR profile comprising unique CDR3 amino acid sequences. In some embodiments, the method further comprises an initial step of obtaining a sample comprising T cells from the subject. In further embodiments, the TCR profile is then compared against a database of previously identified diagnostic public T cell receptor sequences that known to be statistically significantly associated with CMV infection. In some embodiments, a CMV burden score for the subject is then generated by determining the proportion of unique TCR sequences in the profile of the subject that match the public TCR sequences in the database. In further embodiments, the calculated CMV burden score is input into a logistic regression model that compares CMV burden and CMV infection status from a plurality of subjects of known CMV infection status. In one embodiment, an estimated probability of CMV infection status is then determined for the subject as the logistic regression model output.

In some embodiments, the database of public T cell sequences are determined to be statistically associated by obtaining unique TCR sequences from a group of subjects with CMV infection and a group of subjects without CMV infection. In one embodiment, each unique TCR sequence is subjected to a one-tailed Fisher exact test based on the presence or absence of each TCR sequence in subjects of known CMV infection status. In one embodiment, the null hypothesis being that each TCR sequence is no more common in subjects with a CMV infection than in subjects without a CMV infection. In one embodiment, a nominal p value threshold is set, and the false discovery rate (FDR) is controlled by permutation of CMV infection status in each subject to generate an empirical null distribution of p-values. In a particular embodiment, a database is then generated that comprises T cell receptor sequences that are statistically significantly shared in subjects with CMV infection.

In some embodiments, the nominal p-value threshold is less than or equal to 1.0*10⁻⁴.

In some embodiments, the statistically identified unique TCR sequences that correlate with the presence or absence of CMV infection comprise one or more of SEQ ID NOs: 1 to 142. In some embodiments, the statistically identified unique TCR sequences that correlate with the presence or absence of CMV infection comprise SEQ ID NOs: 1 to 142.

In some embodiments, the step of determining the at least 100,000 unique TCRβ sequences from the sample includes amplifying the rearranged nucleic acids encoding the TCRβ CDR3 region in a multiplex PCR reaction with a mixture of forward primers specific to TCR Vβ gene segments and reverse primers specific to TCR Jβ gene segments. In some embodiments, the reads of the amplified nucleic acids are sequenced, and the sequence reads are processed to remove errors in the primary sequence of each read and to compress the data. In particular embodiments, a nearest neighbor algorithm is applied to collapse the data into unique sequences by merging closely related sequences to remove both PCR and sequencing errors.

The method also includes determining an HLA association for each unique TCR sequence in the TCR profile. In some embodiments, the unique TCR sequence is associated with an HLA-A and/or an HLA-B allele.

The method of the invention also comprises steps for predicting a human leukocyte antigen (HLA) allele status of a subject, comprising (a) determining an immune receptor profile of unique T-cell receptor (TCR) rearranged DNA sequences for each of a plurality of subjects, each subject having a known HLA allele status; (b) categorizing the plurality of subjects based on (i) said known HLA allele status of the subject and (ii) a presence or absence in the subject's immune receptor profile of a feature comprising a unique TCR rearranged DNA sequence; (c) determining a statistical score for the association between a set of features and a positive HLA allele status based on (b); (d) training a machine learning model using said set of features to define a set of classifiers for each HLA allele status; (e) inputting one or more unique TCR rearranged DNA sequences of a subject with an unknown HLA allele status into said machine learning model to identify one or more features that match the set of classifiers; and (f) predicting an HLA allele status of said subject based on said one or more matched features.

The method comprises determining an immune receptor profile by determining the total number of unique TCR sequences and the frequency of each unique TCR sequence. The method also comprises determining a statistical score comprising determining a p-value using a Fisher exact two-tailed test.

In some embodiments, the method includes determining a cutoff p-value for identifying a set of features that are significantly associated with an HLA allele status. In another embodiment, the method includes determining a false discovery rate (FDR) of the association of a feature with an HLA allele status. In other embodiments, the method includes determining a number of false-positive associations between said feature and said HLA allele status.

In some embodiments, the method includes training a machine learning model by training a logistic regression model using said set of identified features and said known HLA allele statuses of each subject. In one embodiment, the method includes training a machine learning model comprises performing a leave-one out cross validation method. In another embodiment, the method comprises performing said leave-one out cross validation method for multiple rounds. In another embodiment, the prediction is at least 80% accurate or is at least 90% accurate.

In certain embodiments, the TCR rearranged DNA sequence is a TCRA, TCRB, TCRG or TCRD rearranged DNA sequence. In other embodiments, the HLA allele is a HLA-A2 allele or a HLA-24 allele.

### BRIEF DESCRIPTION OF THE DRAWINGS

A better understanding of the novel features of the invention and advantages of the present invention will be obtained by reference to the following description that sets forth illustrative embodiments, in which the principles of the invention are utilized, and the accompanying drawings of which:

FIG. 1 depicts eleven distinct CDR3β VDJ recombination events, while still yielding the same nucleotide sequence, and ultimately the same CDR3β amino acid sequence (SEQ ID NO:149). The nucleotide sequences on the left side of FIG. 1 are, in order from top to bottom, SEQ ID NO:143, SEQ ID NO: 144, SEQ ID NO: 144, SEQ ID NO: 144, SEQ ID NO: 144, SEQ ID NO: 144, SEQ ID NO: 145, SEQ ID NO: 146, SEQ ID NO: 146, SEQ ID NO: 147, SEQ ID NO: 148.

FIGS. 2A, 2B and 2C provide an overview of the method for predicting disease status of an individual, according to one embodiment of the invention. FIG. 2A shows a dataset of peripheral blood samples from 640 healthy subjects (287 CMV− and 353 CMV+), which were analyzed by high-throughput TCR immune receptor profiling. In FIG. 2B, unique TCRß sequences were identified that were present in significantly more CMV+ subjects than CMV− subjects, controlling for false determination rate (FDR) by permutation of CMV status. Presence of these CMV-associated TCRβ sequences was used to build a classification model. The top panel of FIG. 2B depicts CASSLIGVSSYNEQFF (SEQ ID NO: 12). In FIG. 2C, the classification model was tested using an exhaustive leave-one-out cross-validation, in which one sample was held out of the calculations, and the process was repeated from the beginning. The resulting classification model was used to predict the CMV status of the holdout subject.

FIG. 3A depicts a graphical representation of a subject data set, where 640 subjects were specifically phenotyped for HLA type and CMB status for eligibility as an HCT donor, and segregated between 287 CMV+ donors and 353 CMV− donors. There were roughly equal numbers of seropositive and seronegative samples for the investigation of public T cell responses.

FIG. 3B shows demographic characteristics of the subjects included in this study, classified by CMV status.

FIG. 4 depicts a test for CMV-association of TCRs, where the TCR amino acid sequence CASSLIGVSSYNEQFF (SEQ ID NO:12) was selected and identified in 27 of the CMV+ subjects, and only 2 of the CMV− subjects, with a p-value of 2.8E⁻⁰⁸.

FIG. 5 depicts exemplary TCR amino acid sequences identified in CMV+ subjects with a p-value threshold chosen at 10⁻⁴. In order from top to bottom, these are SEQ ID NOs: 1-5. One can see that at a lower p-value, larger numbers of CMB-associated TCRβ sequences can be identified.

FIG. 6 depicts the CMV burden, which is represented as the proportion of unique TCRs in each subject that are significantly CMV-associated versus those TCRs that are not significantly CMV-associated.

FIG. 7 depicts the cross-validation method, which leaves out one of the initial 640 samples, then the database of CMV-specific TCRβ sequences and associated statistics is retrained in an unsupervised fashion to eliminate bias, and the CMV serostatus of the left-out sample is classified. FIG. 7 depicts CASSLIGVSSYNEQFF (SEQ ID NO:12).

FIG. 8 depicts the results of the cross-validation method shown for all subjects and cross-validated subjects. The data is presented as the area under receiver operating characteristic (AUROC) in the y-axis versus the p-value across the x-axis. The results of the cross-validation method are shown as a plot of the false positive rate versus the true positive rate. FIG. 8 (top graph) shows data for the classification performance of all and the cross-validation (CV) datasets for each p-value threshold, measured as the area under the ROC curve (AUROC). The number above each set of data points corresponds to the number of CMV-associated TCRβ identified at that p-value threshold, and the rectangle indicates the dataset selected for downstream analysis (p-value=10⁻⁴). FIG. 8 (bottom graph) also shows a false discovery rate (FDR) estimated for each p-value threshold used in the identification of significantly CMV-associated TCRβ sequences, using permutations of CMV status. The best performance is seen at a p-value of 10⁻⁴, which corresponds to an estimated FDR of ˜20%, resulting in the identification of a set of 142 TCRβ sequences that were significantly associated with positive CMV status (listed in Table 1).

FIG. 9 shows the ROC curves for both the all and the cross-validation datasets.

FIGS. 10A and 10B show HLA-restriction of CMV-associated TCRβ sequences. FIG. 10A shows the distribution of HLA-A alleles in this cohort. FIG. 10B shows the distribution of HLA-B alleles in this cohort. FIGS. 10A and 10B depict the following sequences: CASSLAPGATNEKLFF (SEQ ID NO:8), CASSLIGVSSYNEQFF (SEQ ID NO:12), CASSPSRNTEAFF (SEQ ID NO:73), CASSLQAGANEQFF (SEQ ID NO:119), and CASASANYGYTF (SEQ ID NO:118).

FIGS. 11A and 11B show the incidence of previously reported CMV-reactive TCRβ sequences in this cohort. FIG. 11A shows the incidence of each such TCRβ sequence in the cohort of 640 subjects plotted along the horizontal axis by decreasing total incidence, with the incidence in CMV+ subjects above the horizontal and the incidence in CMV− subjects below the horizontal. FIG. 11B shows a histogram of incidence of these TCRβ sequences in the cohort of 640 subjects plotted for each group of sequences.

FIG. 12 shows the concordance of TCRß sequences in the cohort as compared to those in the literature. FIG. 12 depicts the following sequences: CASSLAPGATNEKLFF (SEQ ID NO:8), CASSLIGVSSYNEQFF (SEQ ID NO:12), CASSPSRNTEAFF (SEQ ID NO: 73), CASSLQAGANEQFF (SEQ ID NO:119), and CASASANYGYTF (SEQ ID NO: 118).

FIG. 13 depicts an overview of one embodiment of the method. FIG. 13 depicts CSARDRGIGNTIYF (SEQ ID NO:152).

FIG. 14 depicts feature selection using a two-tailed Fisher exact test to determine statistical significance of the association between a feature (unique TCRB sequence) and HLA allele status (HLA-A2+ or HLA-A2−), according to an embodiment of the invention. An exemplary list is shown of features (unique TCRB sequences) and the number of subjects who have a particular feature and whether the subject is positive or negative for the HLA-A2 allele. FIG. 14 depicts, in order from top to bottom, SEQ ID NOs: 150-161.

FIG. 15 depicts steps for feature selection, according to an embodiment of the invention. A p value is selected as a cutoff for identifying a set of “Feature TCRs” from the entire list of possible TCR sequences. Defining a p-value threshold and permuting the allele status across individuals provides an estimate of false discovery rate. This is performed for each HLA allele, resulting in a set of allele-associated TCRβ sequences for each HLA allele. A p value cutoff of p≤10⁻⁴ and an FDR of 0.1 was used to identify 288 TCRB sequences that are positively associated with HLA-A2. For each of the allele-associated TCRB sequences, the frequency of the sequence is also determined in each subject. FIG. 15 depicts, in order from left to right, SEQ ID NOs: 162 and 163.

FIG. 16 depicts a machine learning process for fitting a logistic regression model, according to an embodiment of the invention.

FIG. 17 depicts an exhaustive leave-one-out cross validation, according to an embodiment of the invention.

FIG. 18 shows the results of the cross-validation experiment and illustrates the accuracy of the method.

### DETAILED DESCRIPTION OF THE INVENTION

The invention provides methods for predicting and diagnosing infectious disease in a subject that are disease-specific (specific for each particular disease state), have a universal platform (do not require different processes/reagents for each disease state), and multiplexed (are able to assay multiple disease states simultaneously.

In one embodiment, the method includes steps for high-throughput immunosequencing of rearranged TCR genes in healthy subjects with known CMV status. Using the immunosequencing results, the method includes searching for TCRβ sequences that are present in multiple subjects, and identifying a set of TCRβ sequences that are significantly associated with positive CMV status. The method also includes calculating a p-value for the association of each TCRβ sequence with CMV status using a Fisher exact test, controlling the false discovery rate (FDR) by permutation of the CMV status, and identifying a list of CMV-associated TCRβ sequences (for a certain FDR and p-value). A CMV score is calculated for each subject as the proportion of all that subject's TCRβ sequences that are represented in the catalog of CMV-associated TCRβ sequences. The CMV score is used to distinguish between CMV+ and CMV− subjects.

Infectious agents include pathogens, viruses, bacteria, parasites and/or microorganisms. In some embodiments, viruses include, but are not limited to, members of the herpes virus family (such as herpes simplex viruses 1 and 2, varicella-zoster virus, EBV (Epstein-Barr virus), human cytomegalovirus (CMV), human herpesvirus 6, human herpesvirus 7, and Kaposi's sarcoma-associated herpesvirus), Hepatitis B virus (HBV), Hepatitis C virus (HCV), human immunodeficiency viruses (HIV) I and II, influenza A virus, influenza B virus, respiratory syncytial viruses (RSV) A and B, and human metapneumovirus (MPV). Other examples include human T-cell lymphocytotrophic virus, human papillomaviruses, orthomyxo viruses, paramyxo viruses, adenoviruses, corona viruses, rhabdo viruses, polio viruses, toga viruses, bunya viruses, arena viruses, rubella viruses, reo viruses, Norovirus, human metapneumovirus (MPV), West Nile virus, Yellow fever virus, Rabies virus, Rhinovirus, Rift Valley fever virus, Marburg virus, mumps virus, measles virus, human papilloma virus (HPV), Ebola virus, Colorado tick fever virus (CTFV), and/or rhinoviruses.

Other infectious organisms include Escherichia coli, Salmonella, Shigella, Campylobacter, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Clostridium difficile, Gardnerella, Trichomonas vaginalis, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, Acitnomycetes and/or Acinetobacter.

In still other embodiments, fungal infectious agents include, but are not limited to, Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and/or Maduromycosis.

In more embodiments, parasitic agents include, but are not limited to, Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidium, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, Trematodes, Diphyllobothrium latum, Taenia spp., Pneumocystis carinii, and/or Necator americanis.

The major histocompatibility complex (MHC) is a set of cell surface molecules encoded by a large gene family which controls a major part of the immune system in all vertebrates. The major function of major histocompatibility complexes is to bind to peptide fragments derived from pathogens and display them on the cell surface for recognition by the appropriate T-cells.

The major histocompatibility complex (MHC) contains two types of polymorphic MHC genes, the class I and class II genes, which encode two groups of structurally distinct but homologous proteins, and other nonpolymorphic genes whose products are involved in antigen presentation.

The human MHC is called human leukocyte antigen (HLA). HLA proteins are encoded by genes of the MHC. HLA class I antigens include HLA-A, HLA-B, and HLA-C. HLA class II antigens include HLA-DR, HLA-DQ, HLA-DP, HLA-DM, HLA-DOA, HLA-DOB. HLA's corresponding to MHC class I (A, B, and C) present peptides from inside the cell. HLA's corresponding to MHC class II (DP, DM, DOA, DOB, DQ, and DR) present antigens from outside of the cell to T-lymphocytes. MHC molecules mediate the binding of a given T cell receptor to a given antigen, so MHC polymorphism across individuals modulates the T cell response to a given antigen. Clinical applications of HLA typing include vaccine trials, disease associations, adverse drug reactions, platelet transfusion, and transplantation of organs and stem cells.

There are numerous alleles at each HLA gene locus (A1, A2, A3, etc.). Each person inherits one complete set of HLA alleles (haplotype) from each parent, and this combination of encoded proteins constitutes a person's HLA type (e.g., different antigens of A23, A31, B7, B44, C7, C8, DR4, DR7, DQ2, DQ7, DP2, DP3). There are more than 50,000 different known HLA types.

As used herein, adaptive immune receptor (AIR) refers to an immune cell receptor, e.g., a T cell receptor (TCR) or an Immunoglobulin (Ig) receptor found in mammalian cells.

In certain embodiments, the adaptive immune receptor is encoded by a TCRB, TCRG, TCRA, TCRD, IGH, IGK, and IGL gene or gene segment.

The term “primer,” as used herein, refers to an oligonucleotide sequence capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature.

As used herein, the term “gene” refers to the segment of DNA involved in producing a polypeptide chain, such as all or a portion of a TCR or Ig polypeptide (e.g., a CDR3-containing polypeptide); it includes regions preceding and following the coding region “leader and trailer” as well as intervening sequences (introns) between individual coding segments (exons), and can also include regulatory elements (e.g., promoters, enhancers, repressor binding sites and the like), and can also include recombination signal sequences (RSSs), as described herein.

The nucleic acids of the present embodiments, also referred to herein as polynucleotides, and including oligonucleotides, can be in the form of RNA or in the form of DNA, including cDNA, genomic DNA, and synthetic DNA. The DNA can be double-stranded or single-stranded, and if single stranded can be the coding strand or non-coding (anti-sense) strand. A coding sequence which encodes a TCR or an immunoglobulin or a region thereof (e.g., a V region, a D segment, a J region, a C region, etc.) for use according to the present embodiments can be identical to the coding sequence known in the art for any given TCR or immunoglobulin gene regions or polypeptide domains (e.g., V-region domains, CDR3 domains, etc.), or can be a different coding sequence, which, as a result of the redundancy or degeneracy of the genetic code, encodes the same TCR or immunoglobulin region or polypeptide.

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques can be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to.” By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and can or cannot be present depending upon whether or not they affect the activity or action of the listed elements.

In this specification and the appended claims, the singular forms “a,” “an” and “the” include plural references unless the content clearly dictates otherwise.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

### Methods of the Invention

### Cells

A sample containing lymphoid nucleic acid molecules (genomic DNA, cDNA or alternatively, messenger RNA) from a subject can be obtained. The subject is a mammalian subject, such as a human.

Lymphocytes (B cells and/or T cells) can be obtained from a biological sample, such as from a variety of tissue and biological fluid samples. These include but are not limited to bone marrow, thymus, lymph glands, lymph nodes, peripheral tissues and blood, or solid tissue samples. Any peripheral tissue can be sampled for the presence of B and T cells and is therefore contemplated for use in the methods described herein. Peripheral blood mononuclear cells (PBMC) are isolated by techniques known to those of skill in the art, e.g., by Ficoll-Hypaque® density gradient separation. In certain embodiments, whole PBMCs are used for analysis.

### Nucleic Acid Extraction

Total genomic DNA can be extracted from cells by methods known to those of skill in the art. Examples include using the QIAamp® DNA blood Mini Kit (QIAGEN®) or a Qiagen DNeasy Blood extraction Kit (Qiagen, Gaithersburg, MD, USA). The approximate mass of a single haploid genome is 3 pg. Preferably, at least 100,000 to 200,000 cells are used for analysis of diversity, i.e., about 0.6 to 1.2 μg DNA from diploid T cells. Using PBMCs as a source, the number of T cells can be estimated to be about 30% of total cells. Alternatively, total nucleic acid can be isolated from cells, including both genomic DNA and mRNA. In other embodiments, cDNA is transcribed from mRNA and then used as templates for amplification.

### Multiplex Quantitative PCR

Multiplex quantitative PCR was performed as described herein and in Robins et al., 2009 Blood 114, 4099; Robins et al., 2010 Sci. Translat. Med. 2: 47ra64; Robins et al., 2011 J. Immunol. Meth. doi: 10.1016/j.jim.2011.09. 001; Sherwood et al. 2011 Sci. Translat. Med. 3:90ra61; US 2012/0058902, US 2010/0330571, WO/2010/151416, WO/2011/106738 (PCT/US2011/026373), US 2015/0299785, WO2012/027503 (PCT/US2011/049012), US 2013/0288237, U.S. Pat. Nos. 9,181,590, 9,181,591, and US 2013/0253842, which are incorporated by reference in their entireties. The present methods involve a multiplex PCR method using a set of forward primers that specifically hybridize to V segments and a set of reverse primers that specifically hybridize to the J segments of a TCR or Ig locus, where a multiplex PCR reaction using the primers allows amplification of all the possible VJ (and VDJ) combinations within a given population of T or B cells.

Exemplary V segment primers and J segment primers are described in US 2012/0058902, US 2010/033057, WO/2010/151416, WO/2011/106738 (PCT/US2011/026373), US 2015/0299785, WO2012/027503 (PCT/US2011/049012), US 2013/0288237, U.S. Pat. Nos. 9,181,590, 9,181,591, US 2013/0253842, WO 2013/188831 (PCT/US2013/045994), which are incorporated by reference in their entireties.

A multiplex PCR system can be used to amplify rearranged adaptive immune cell receptor loci. In certain embodiments, the CDR3 region is amplified from a TCRA, TCRB, TCRG or TCRD CDR3 region or similarly from an IgH or IgL (lambda or kappa) locus. A plurality of V-segment and J-segment primers are used to amplify substantially all (e.g., greater than 90%, 91%, 92%, 93%, 94%, 95%, 96%, 97%, 98% or 99%) rearranged adaptive immune receptor CDR3-encoding regions to produce a multiplicity of amplified rearranged DNA molecules. In certain embodiments, primers are designed so that each amplified rearranged DNA molecule is less than 600 nucleotides in length, thereby excluding amplification products from non-rearranged adaptive immune receptor loci.

In some embodiments, two pools of primers are used in a single, highly multiplexed PCR reaction. The “forward” pool of primers can include a plurality of V segment oligonucleotide primers and the reverse pool can include a plurality of J segment oligonucleotide primers. In some embodiments, there is a primer that is specific to (e.g., having a nucleotide sequence complementary to a unique sequence region of) each V region segment and to each J region segment in the respective TCR or Ig gene locus. In other embodiments, a primer can hybridize to one or more V segments or J segments, thereby reducing the number of primers required in the multiplex PCR. In certain embodiments, the J-segment primers anneal to a conserved sequence in the joining (“J”) segment.

Each primer can be designed such that a respective amplified DNA segment is obtained that includes a sequence portion of sufficient length to identify each J segment unambiguously based on sequence differences amongst known J-region encoding gene segments in the human genome database, and also to include a sequence portion to which a J-segment-specific primer can anneal for resequencing. This design of V- and J-segment-specific primers enables direct observation of a large fraction of the somatic rearrangements present in the adaptive immune receptor gene repertoire within an individual.

In one embodiment, the present disclosure provides a plurality of V-segment primers and a plurality of J-segment primers. The plurality of V-segment primers and the plurality of J-segment primers amplify all or substantially all combinations of the V- and J-segments of a rearranged immune receptor locus. In some embodiments, the method provides amplification of substantially all of the rearranged AIR sequences in a lymphoid cell and is capable of quantifying the diversity of the TCR or IG repertoire of at least 10⁶, 10⁵, 10⁴, or 10³ unique rearranged AIR sequences in a sample. “Substantially all combinations” can refer to at least 80%, 85%, 90%, 91%, 92%, 93%, 94%, 95%, 96%, 97%, 98%, 99% or more of all the combinations of the V- and J-segments of a rearranged immune receptor locus. In certain embodiments, the plurality of V-segment primers and the plurality of J-segment primers amplify all of the combinations of the V- and J-segments of a rearranged immune receptor locus.

In general, a multiplex PCR system can use 2, 3, 4, 5, 6, 7, 8, 9, 10, 11, 12, 13, 14, 15, 16, 17, 18, 19, 20, 21, 22, 23, 24, or 25, and in certain embodiments, at least 26, 27, 28, 29, 30, 31, 32, 33, 34, 35, 36, 37, 38, or 39, and in other embodiments 40, 41, 42, 43, 44, 45, 46, 47, 48, 49, 50, 51, 52, 53, 54, 55, 56, 57, 58, 59, 60, 65, 70, 75, 80, 85, or more forward primers, in which each forward primer specifically hybridizes to or is complementary to a sequence corresponding to one or more V region segments. The multiplex PCR system also uses at least 2, 3, 4, 5, 6, or 7, and in certain embodiments, 8, 9, 10, 11, 12 or 13 reverse primers, or 14, 15, 16, 17, 18, 19, 20, 21, 22, 23, 24, or 25 or more primers, in which each reverse primer specifically hybridizes to or is complementary to a sequence corresponding to one or more J region segments. Various combinations of V and J segment primers can be used to amplify the full diversity of TCR and IG sequences in a repertoire. For details on the multiplex PCR system, including primer oligonucleotide sequences for amplifying TCR and IG sequences, see, e.g., Robins et al., 2009 Blood 114, 4099; Robins et al., 2010 Sci. Translat. Med. 2: 47ra64; Robins et al., 2011. J. Immunol. Meth. doi: 10.1016/j.jim.2011.09. 001; Sherwood et al. 2011 Sci. Translat. Med. 3: 90ra61; US 2012/0058902, US 2010/033057, WO/2010/151416, WO/2011/106738 (PCT/US2011/026373), US 2015/0299785, WO2012/027503 (PCT/US2011/049012), US 2013/0288237, U.S. Pat. Nos. 9,181,590, 9,181,591, US 2013/0253842, WO 2013/188831 (PCT/US2013/045994), which is each incorporated by reference in its entirety.

Oligonucleotides or polynucleotides that are capable of specifically hybridizing or annealing to a target nucleic acid sequence by nucleotide base complementarity can do so under moderate to high stringency conditions. In one embodiment, suitable moderate to high stringency conditions for specific PCR amplification of a target nucleic acid sequence can be between 25 and 80 PCR cycles, with each cycle consisting of a denaturation step (e.g., about 10-30 seconds(s) at greater than about 95° C.), an annealing step (e.g., about 10-30 s at about 60-68° C.), and an extension step (e.g., about 10-60 s at about 60-72° C.), optionally according to certain embodiments with the annealing and extension steps being combined to provide a two-step PCR. As would be recognized by the skilled person, other PCR reagents can be added or changed in the PCR reaction to increase specificity of primer annealing and amplification, such as altering the magnesium concentration, optionally adding DMSO, and/or the use of blocked primers, modified nucleotides, peptide-nucleic acids, and the like.

A primer is preferably a single-stranded DNA. The appropriate length of a primer depends on the intended use of the primer but typically ranges from 6 to 50 nucleotides, or in certain embodiments, from 15-35 nucleotides in length. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

In some embodiments, the V- and J-segment primers are used to produce a plurality of amplicons from the multiplex PCR reaction. In certain embodiments, the amplicons range in size from 10, 20, 30, 40, 50, 75, 100, 200, 300, 400, 500, 600, 700, 800 or more nucleotides in length. In preferred embodiments, the amplicons have a size between 50-600 nucleotides in length.

According to non-limiting theory, these embodiments exploit current understanding in the art (also described above) that once an adaptive immune cell (e.g., a T or B lymphocyte) has rearranged its adaptive immune receptor-encoding (e.g., TCR or Ig) genes, its progeny cells possess the same adaptive immune receptor-encoding gene rearrangement, thus giving rise to a clonal population (clones) that can be uniquely identified by the presence therein of rearranged (e.g., CDR3-encoding) V- and J-gene segments that can be amplified by a specific pairwise combination of V- and J-specific oligonucleotide primers as herein disclosed.

In some embodiments, the V segment primers and J segment primers each include a second sequence at the 5′-end of the primer that is not complementary to the target V or J segment. The second sequence can comprise an oligonucleotide having a sequence that is selected from (i) a universal adaptor oligonucleotide sequence, and (ii) a sequencing platform-specific oligonucleotide sequence that is linked to and positioned 5′ to a first universal adaptor oligonucleotide sequence. Examples of universal adaptor oligonucleotide sequences can be pGEX forward and pGEX reverse adaptor sequences.

In some embodiments, the resulting amplicons using the V-segment and J-segment primers described above include amplified V and J segments and the universal adaptor oligonucleotide sequences. The universal adaptor sequence can be complementary to an oligonucleotide sequence found in a tailing primer. Tailing primers can be used in a second PCR reaction to generate a second set of amplicons. In some embodiments, tailing primers can have the general formula:

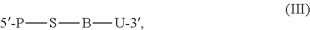

wherein P comprises a sequencing platform-specific oligonucleotide,
wherein S comprises a sequencing platform tag-containing oligonucleotide sequence;
wherein B comprises an oligonucleotide barcode sequence and wherein said oligonucleotide barcode sequence can be used to identify a sample source, and
wherein U comprises a sequence that is complementary to the universal adaptor oligonucleotide sequence or is the same as the universal adaptor oligonucleotide sequence.

Additional description about universal adaptor oligonucleotide sequences, barcodes, and tailing primers are found in WO 2013/188831 (PCT/US13/45994), which is incorporated by reference in its entirety.

### Amplification Bias Control

Multiplex PCR assays can result in a bias in the total numbers of amplicons produced from a sample, given that certain primer sets are more efficient in amplification than others. To overcome the problem of such biased utilization of subpopulations of amplification primers, methods can be used that provide a template composition for standardizing the amplification efficiencies of the members of an oligonucleotide primer set, where the primer set is capable of amplifying rearranged DNA encoding a plurality of adaptive immune receptors (TCR or Ig) in a biological sample that comprises DNA from lymphoid cells.

In some embodiments, a template composition is used to standardize the various amplification efficiencies of the primer sets. The template composition can comprise a plurality of diverse template oligonucleotides of general formula (I):

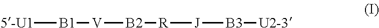

The constituent template oligonucleotides are diverse with respect to the nucleotide sequences of the individual template oligonucleotides. The individual template oligonucleotides can vary in nucleotide sequence considerably from one another as a function of significant sequence variability among the large number of possible TCR or BCR variable (V) and joining (J) region polynucleotides. Sequences of individual template oligonucleotide species can also vary from one another as a function of sequence differences in U1, U2, B (B1, B2 and B3) and R oligonucleotides that are included in a particular template within the diverse plurality of templates.

In certain embodiments, V is a polynucleotide comprising at least 20, 30, 60, 90, 120, 150, 180, or 210, and not more than 1000, 900, 800, 700, 600 or 500 contiguous nucleotides of an adaptive immune receptor variable (V) region encoding gene sequence, or the complement thereof, and in each of the plurality of template oligonucleotide sequences V comprises a unique oligonucleotide sequence.

In some embodiments, J is a polynucleotide comprising at least 15-30, 31-60, 61-90, 91-120, or 120-150, and not more than 600, 500, 400, 300 or 200 contiguous nucleotides of an adaptive immune receptor joining (J) region encoding gene sequence, or the complement thereof, and in each of the plurality of template oligonucleotide sequences J comprises a unique oligonucleotide sequence.

U1 and U2 can be each either nothing or each comprise an oligonucleotide having, independently, a sequence that is selected from (i) a universal adaptor oligonucleotide sequence, and (ii) a sequencing platform-specific oligonucleotide sequence that is linked to and positioned 5′ to the universal adaptor oligonucleotide sequence.

B1, B2 and B3 can be each either nothing or each comprise an oligonucleotide B that comprises a first and a second oligonucleotide barcode sequence of 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900 or 1000 contiguous nucleotides (including all integer values therebetween), wherein in each of the plurality of template oligonucleotide sequences B comprises a unique oligonucleotide sequence in which (i) the first barcode sequence uniquely identifies the unique V oligonucleotide sequence of the template oligonucleotide and (ii) the second barcode sequence uniquely identifies the unique J oligonucleotide sequence of the template oligonucleotide.

R can be either nothing or comprises a restriction enzyme recognition site that comprises an oligonucleotide sequence that is absent from V, J, U1, U2, B1, B2 and B3.

Methods are used with the template composition for determining non-uniform nucleic acid amplification potential among members of a set of oligonucleotide amplification primers that are capable of amplifying productively rearranged DNA encoding one or a plurality of adaptive immune receptors in a biological sample that comprises DNA from lymphoid cells of a subject. The method can include the steps of: (a) amplifying DNA of a template composition for standardizing amplification efficiency of an oligonucleotide primer set in a multiplex polymerase chain reaction (PCR) that comprises: (i) the template composition (I) described above, wherein each template oligonucleotide in the plurality of template oligonucleotides is present in a substantially equimolar amount; (ii) an oligonucleotide amplification primer set that is capable of amplifying productively rearranged DNA encoding one or a plurality of adaptive immune receptors in a biological sample that comprises DNA from lymphoid cells of a subject.

The primer set can include: (1) in substantially equimolar amounts, a plurality of V-segment oligonucleotide primers that are each independently capable of specifically hybridizing to at least one polynucleotide encoding an adaptive immune receptor V-region polypeptide or to the complement thereof, wherein each V-segment primer comprises a nucleotide sequence of at least 15 contiguous nucleotides that is complementary to at least one functional adaptive immune receptor V region-encoding gene segment and wherein the plurality of V-segment primers specifically hybridize to substantially all functional adaptive immune receptor V region-encoding gene segments that are present in the template composition, and (2) in substantially equimolar amounts, a plurality of J-segment oligonucleotide primers that are each independently capable of specifically hybridizing to at least one polynucleotide encoding an adaptive immune receptor J-region polypeptide or to the complement thereof, wherein each J-segment primer comprises a nucleotide sequence of at least 15 contiguous nucleotides that is complementary to at least one functional adaptive immune receptor J region-encoding gene segment and wherein the plurality of J-segment primers specifically hybridize to substantially all functional adaptive immune receptor J region-encoding gene segments that are present in the template composition.

The V-segment and J-segment oligonucleotide primers are capable of promoting amplification in said multiplex polymerase chain reaction (PCR) of substantially all template oligonucleotides in the template composition to produce a multiplicity of amplified template DNA molecules, said multiplicity of amplified template DNA molecules being sufficient to quantify diversity of the template oligonucleotides in the template composition, and wherein each amplified template DNA molecule in the multiplicity of amplified template DNA molecules is less than 1000, 900, 800, 700, 600, 500, 400, 300, 200, 100, 90, 80 or 70 nucleotides in length.

The method also includes steps of: (b) sequencing all or a sufficient portion of each of said multiplicity of amplified template DNA molecules to determine, for each unique template DNA molecule in said multiplicity of amplified template DNA molecules, (i) a template-specific oligonucleotide DNA sequence and (ii) a relative frequency of occurrence of the template oligonucleotide; and (c) comparing the relative frequency of occurrence for each unique template DNA sequence from said template composition, wherein a non-uniform frequency of occurrence for one or more template DNA sequences indicates non-uniform nucleic acid amplification potential among members of the set of oligonucleotide amplification primers.

Further description about bias control methods are provided in US 2013/0253842, U.S. Pat. No. 9,150,905, US 2015/0203897, and WO 2013/169957 (PCT/US2013/040221), which are incorporated by reference in their entireties.

### Sequencing

Sequencing can be performed using any of a variety of available high throughput single molecule sequencing machines and systems. Illustrative sequence systems include sequence-by-synthesis systems, such as the Illumina Genome Analyzer and associated instruments (Illumina HiSeq) (Illumina, Inc., San Diego, CA), Helicos Genetic Analysis System (Helicos BioSciences Corp., Cambridge, MA), Pacific Biosciences PacBio RS (Pacific Biosciences, Menlo Park, CA), or other systems having similar capabilities. Sequencing is achieved using a set of sequencing platform-specific oligonucleotides that hybridize to a defined region within the amplified DNA molecules. The sequencing platform-specific oligonucleotides are designed to sequence up amplicons, such that the V- and J-encoding gene segments can be uniquely identified by the sequences that are generated. See, e.g., US 2012/0058902; US 2010/033057; WO 2011/106738 (PCT/US2011/026373); US 2015/0299785; or WO 2012/027503 (PCT/US2011/049012), which is each incorporated by reference in its entirety.

In some embodiments, the raw sequence data is preprocessed to remove errors in the primary sequence of each read and to compress the data. A nearest neighbor algorithm can be used to collapse the data into unique sequences by merging closely related sequences, to remove both PCR and sequencing errors. See, e.g., US 2012/0058902; US 2010/033057; WO 2011/106738 (PCT/US2011/026373); US 2015/0299785; or WO 2012/027503 (PCT/US2011/049012), which is each incorporated by reference in its entirety.

### PCR Template Abundance Estimation

To estimate the average read coverage per input template in the multiplex PCR and sequencing approach, a set of synthetic TCR (or BCR) templates (as described above) can be used, comprising each combination of Vβ and Jβ gene segments. These synthetic molecules can be those described in general formula (I) above, and in US 2013/0253842, U.S. Pat. No. 9,150,905, US 2015/0203897, and WO 2013/169957 (PCT/US2013/040221), which are incorporated by reference in their entireties.

These synthetic molecules can be included in each PCR reaction at very low concentration so that only some of the synthetic templates are observed. Using the known concentration of the synthetic template pool, the relationship between the number of observed unique synthetic molecules and the total number of synthetic molecules added to reaction can be simulated (this is very nearly one-to-one at the low concentrations that were used). The synthetic molecules allow calculation for each PCR reaction the mean number of sequencing reads obtained per molecule of PCR template, and an estimation of the number of T cells in the input material bearing each unique TCR rearrangement.

### Discovery of Diagnostic Public T Cell Responses

Given a large population of subjects with and without a given infectious disease, public T cell responses diagnostic for the disease state can be determined by applying the following statistical method. An immune receptor repertoire of unique T cell receptor sequences is determined, using the methods described above, for a group of subjects who have been identified as having or not having an infectious disease, such as CMV or small pox. It is possible, then, to determine which of those T cell receptor sequences are significantly more common (i.e., present in more individuals) among subjects with the disease state than in subjects without the disease state. When a common TCR binds to an antigen, it is called a public T cell response. Public T cell responses are specific for a particular disease or antigen, are present in many individuals, and are encoded in a common format (specific rearranged receptor sequences) regardless of disease.

A one-tailed Fisher exact test is used, using presence or absence of T cell receptor sequence in question vs. with or without disease state to construct a 2×2 contingency table (shown below) and with FDR (false discovery rate) controlled using an empirical distribution of null p-values determined by permutations of disease state. Since many clones are unique to a single subject (and consequently unique to either the positive or negative disease status classes), it is vital to control false discovery rate in feature selection to avoid over-fitting to the many spurious associations of unique TCRβs with positive infectious disease status. This process generates a list of T cell receptor sequences significantly more common in subjects with the disease state of interest.

| | Disease+ | Disease− |
|---|---|---|
| clone i present | nᵢ₊ | nᵢ₋ |
| clone i not present | N₊ − nᵢ₊ | N₋ − nᵢ₋ |

### Use of Diagnostic Public T Cell Responses to Infer Disease Status

Given a large population of subjects with and without a given infectious disease, and given a list of diagnostic public T cell responses generated as described above, it is possible to infer disease status in a subject of unknown disease status.

First, a ‘disease burden’ or quantitative measure of the presence and/or abundance of said diagnostic public T cell responses is calculated for each subject (those with known status as well as the subject of unknown status). The disease burden or quantitative measure is the proportion of unique T cell receptor sequences in each subject that are among the list of diagnostic public T cell responses.

Once this measure has been calculated, it is then determined whether the subject of unknown status has a disease burden consistent with known subjects who have the disease state of interest or with known subjects who do not have the disease state of interest. Herein, the method of comparison is to train a logistic regression model of (disease burden vs. presence of disease state) in all subjects of known disease status, and use that model to assign to the subject of unknown status a probability of having the disease status of interest.

In some embodiments, the method comprises a classification model using an exhaustive leave-one-out cross-validation, in which the immune profile dataset from an individual is held out of the calculations, and the process is repeated from the beginning. In one aspect, cross-validation is used to assess the accuracy of a classification model. The resulting classification model is used to predict the infectious disease status of the holdout subject.

These methods can be performed for determining disease status for various types of disease, including but not limited to CMV, EBV, HPV, HIV, small pox, other infectious diseases, or even non-infectious diseases, like autoimmune diseases and malignancies.

### HLA Typing

HLA typing of bone marrow donor subjects was performed according to standard protocol by the Fred Hutchinson Cancer Research Center, and can be performed by methods known to those of skill in the art.

### Method of Determining HLA Status of Unknown Subjects

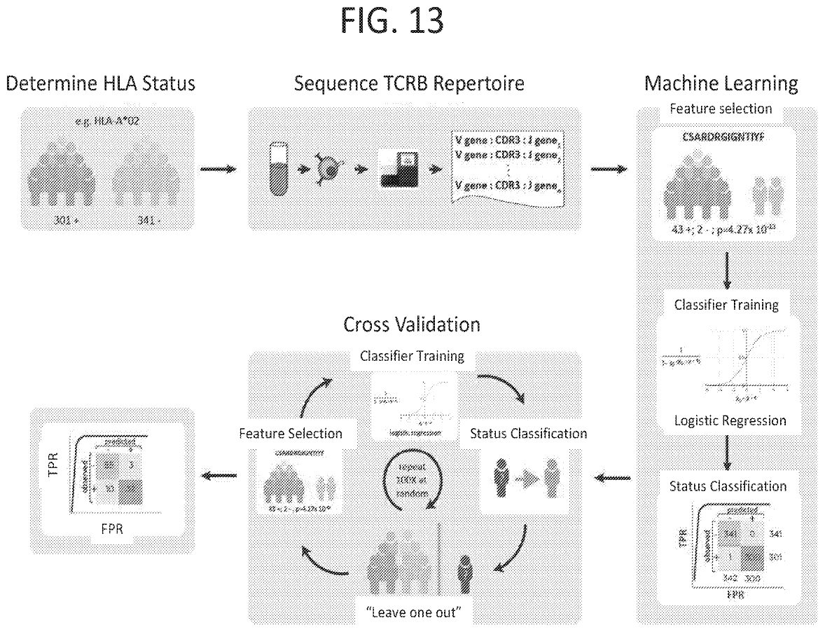

FIG. 13 shows an overview of the method for inferring HLA status. This method can be applied to any HLA allele type and any profile of TCR sequences, including TCRA, TCRB, TCRG, or TCRD gene sequences.

First, the HLA allele type is determined for each subject in a group, according to standard methods in the art for HLA typing. Then, a locus of the subject's immune repertoire is sequenced. For example, the TCRB locus is amplified and sequenced, using the methods described above.

For each subject, the total number of unique TCRB sequences and the frequency of each unique TCRB sequence are determined. For a given HLA allele, it is determined which TCRB sequences are significantly associated with the HLA allele. In one embodiment, for each unique TCRB sequence, it is determined how many subjects who are positive for an HLA allele have the TCRB sequence and how many subjects who are negative for the HLA allele have the TCRB sequence. In addition, it can be determined the number of subjects who are positive for an HLA allele and negative for the TCRB sequence and the number of subjects who are negative for the HLA allele and positive for the TCRB sequence. The table below shows categorization of subjects by the presence or absence of a TCRB sequence and the presence or absence of an HLA allele, HLA-A2.

| | HLA-A2+ | HLA-A2− |
|---|---|---|
| TCRβ sequence i present | nᵢ₊ | nᵢ₋ |
| TCRβ sequence i not present | N₊ − nᵢ₊ | N₋ − nᵢ₋ |

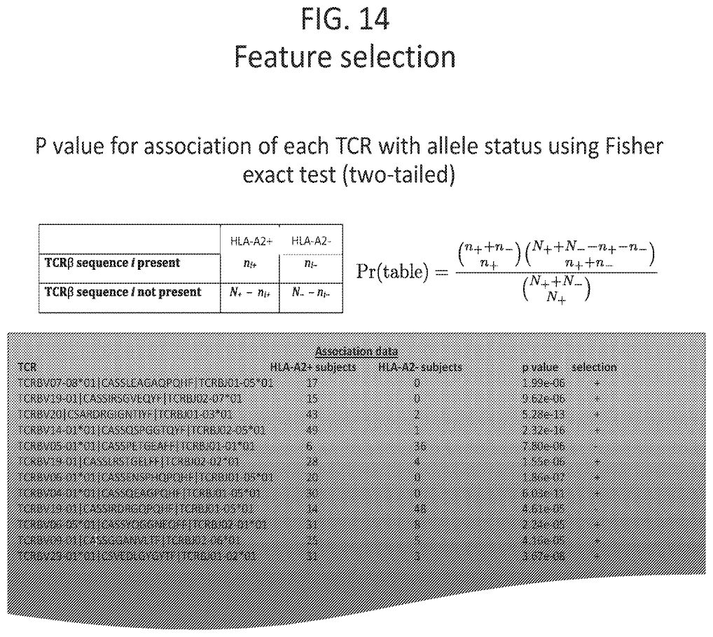

FIG. 14 shows a list of exemplary unique TCRB sequences and the number of subjects who are positive or negative for an HLA-A2 allele that have a particular TCRB sequence.

A p-value is determined for the association of each TCRB sequence with an HLA status using a Fisher exact test (two-tailed). The p value for association of each TCR with allele status using a Fisher exact test (two tailed) is calculated as follows:

$$Pr ( table ) = \frac{( \begin{matrix}{n}_{+} + {n}_{-} \\ {n}_{+}\end{matrix} ) ( \begin{matrix}{N}_{+} + {N}_{-} - {n}_{+} - {n}_{-} \\ {n}_{+} + {n}_{-}\end{matrix} )}{( \begin{matrix}{N}_{+} + {N}_{-} \\ {N}_{+}\end{matrix} )}$$

FIG. 14 also shows exemplary p values that are calculated for the association of a particular TCRB sequence with an HLA type (HLA-A2).

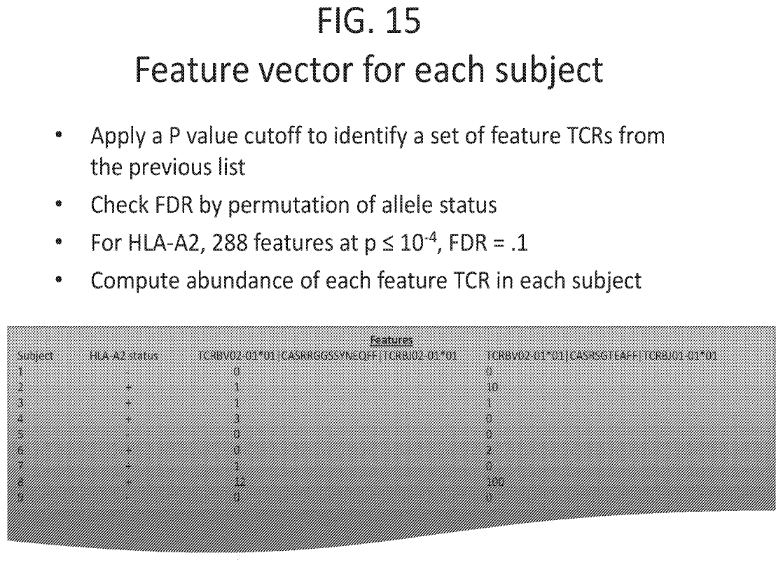

As shown in FIG. 15, a p value is selected as a cutoff for identifying a set of “Feature TCRS” from the entire list of possible TCR sequences. Defining a p-value threshold and permuting the allele status across individuals provides an estimate of false discovery rate. This is performed for each HLA allele, resulting in a set of allele-associated TCRß sequences for each HLA allele present in the training data. In FIG. 15, a p value cutoff of p≤10⁻⁴ and an FDR of 0.1 is used to identify 288 TCRB sequences that are positively associated with HLA-A2. For each of the allele-associated TCRB sequences, the frequency of the sequence is also determined in each subject.

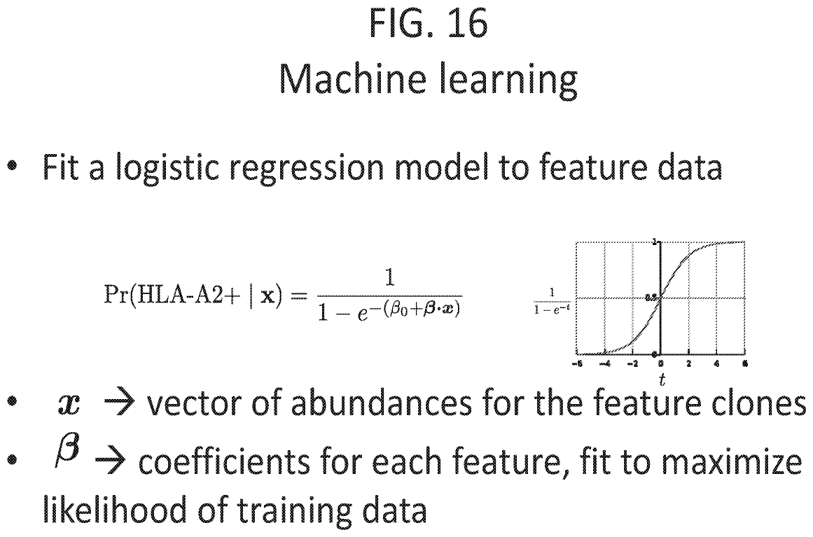

The feature selection step is followed by a machine learning process. As shown in FIG. 16, for each HLA allele, a logistic regression model is trained using the set of feature vectors over all subjects, along with the known status for presence of that allele.

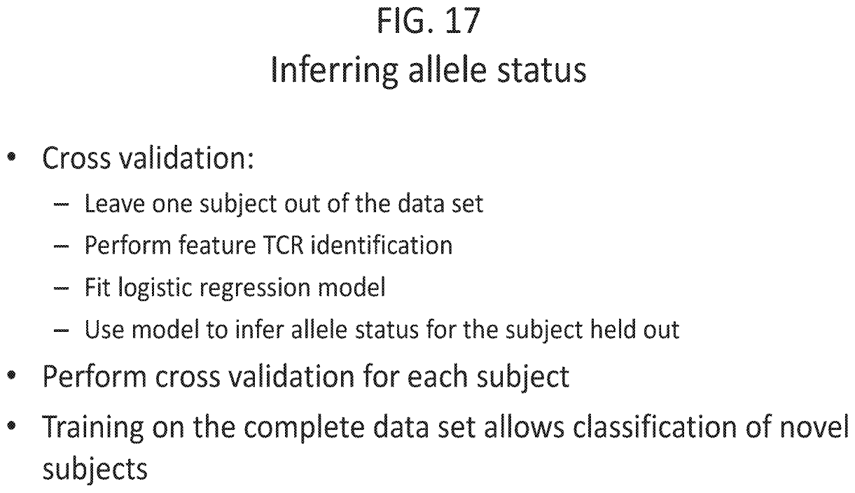

As shown in FIG. 17, an exhaustive leave-one-out cross validation is performed where one subject is removed from the analysis, and the HLA status of the subject is inferred based on feature selection and training from only the remaining subjects. The result is a set of classifiers (one for each HLA allele) that estimate the probability of positive status for each HLA allele, taking as input the feature vector for each allele.

Equipped with these classifiers, the HLA type of a new subject can be assessed by: 1. immune repertoire sequencing, 2. computing feature vectors for each allele, and 3. defining a probability threshold for positive status that will be applied to the output from each classifier. In this manner, an HLA status can be inferred for a new subject with an unknown HLA status.

In an embodiment, given the results of the feature selection scheme, a one-dimension allele score is determined as the fraction of an individual's unique TCRβ sequences that appear in the set of TCRβ sequences associated with a given allele. This single quantity, rather than a vector of features, may also be used for training a classifier and diagnosing novel individuals with similar accuracy.

Additionally, the specific classifier described (logistic regression) can be replaced with any of a number of other binary classifiers and gives substantially similar results. These include: k-nearest neighbors, random forests, artificial neural network, naïve Bayes, and support vector machine.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. Sec, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Identification of Cytomegalovirus (CMV)-Associated TCRs and Classification of CMV Status from Immunosequencing Data

The first aim of the study was to create a comprehensive catalog of CMV-specific T cell receptor sequences. The goal was to identify both common and rare, shared TCRß sequences enriched in CMV seropositive individuals relative to CMV seronegative individuals. CMV was used as a model because it provides an ideal test bed for the development of T cell biomarkers. CMV serostatus is widely available and easy access to near-equal numbers of seropositive (cases) and seronegative (control) individuals among healthy adults provides good statistical power for a rigorous analysis.

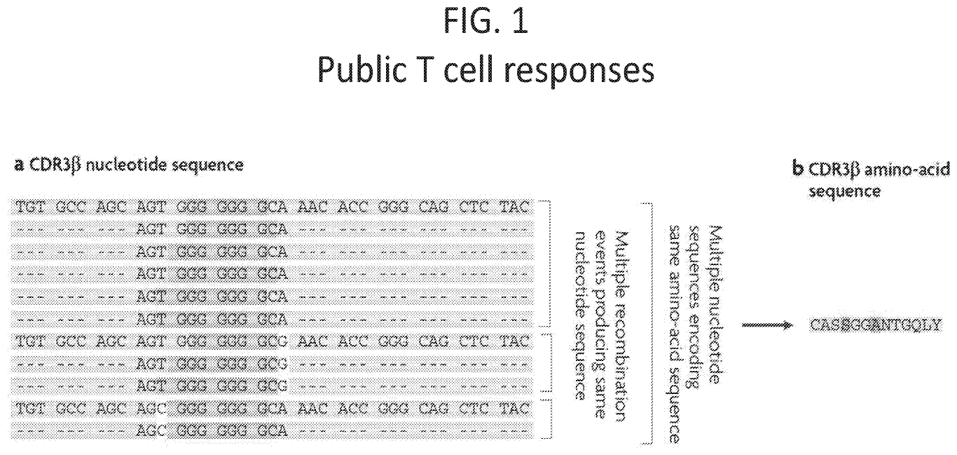

There are many possible TCR sequences generated during VDJ recombination (˜10¹² easily accessible by VDJ recombination). As shown in FIG. 1, different VDJ recombination events may result in the same nucleotide sequence. Moreover, it is possible that VDJ recombination can result in different nucleotide sequences that are translated into the same amino acid sequence. See Venturi, Price, Douek and Davenport 2008. The molecular basis for public T-cell responses? Nature Reviews Immunology. Certain TCRs are common rearrangements and shared by more than one individual. When a common TCR binds to an antigen, it is called a public T cell response. Public T cell responses are specific for a particular disease or antigen, are present in many individuals, and are encoded in a common format (specific rearranged receptor sequences) regardless of disease.

In this study, the public T cell response to cytomegalovirus (CMV) was examined by sequencing of rearranged T cell receptor genes (TCRs) obtained from 640 subjects. CMV is a life-long infection, usually asymptomatic, that afflicts most adults and elicits a robust memory T cell response.

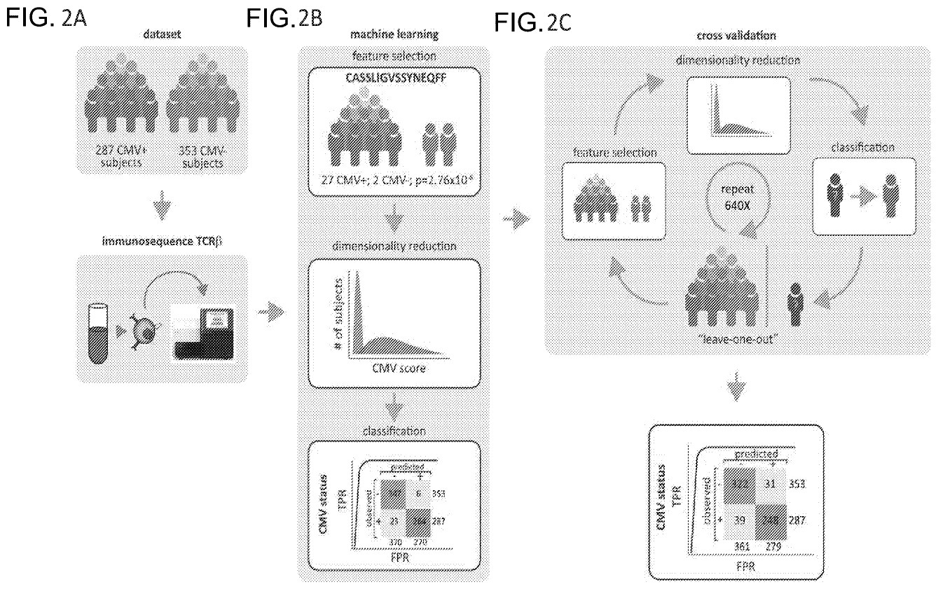

FIGS. 2A, 2B and 2C provide an overview of the method, according to one embodiment of the invention. FIG. 2A shows a dataset of peripheral blood samples from 640 healthy subjects (287 CMV− and 353 CMV+), which were analyzed by high-throughput TCR profiling. In FIG. 2B, unique TCRβ sequences were identified that were present in significantly more CMV+ subjects than CMV− subjects, controlling false determination rate (FDR) by permutation of CMV status. Presence of these CMV-associated TCRβ sequences was used to build a classification model. In FIG. 2C, the classification model was tested using exhaustive leave-one-out cross-validation, in which one sample was held out of the calculations, and the process was repeated from the beginning. The resulting classification model was used to predict the CMV status of the holdout subject.

Human peripheral blood samples were obtained from the Fred Hutchinson Cancer Research Center Research Cell Bank biorepository of healthy bone marrow donors under a protocol following written informed consent approved and supervised by the Fred Hutchinson Cancer Research Center Institutional Review Board. This biorepository houses an inventory of PBMC, B-LCL and DNA from hematopoietic cell transplant (HCT) patients, donors and family members along with cell lines and DNA derived from stem cell transplant patients, donors and selected family members from ˜7,800 stem cell transplant patients and 7,300 donors (from which samples were drawn). The DNA for this study was extracted from peripheral blood of HCT donors and extensively typed for HLA antigens and alleles along with other defining phenotypes needed for donor-patient matching or donor inclusion/exclusion. Among these are cytomegalovirus (CMV), Epstein-Barr virus (EBV), Herpes Simplex Virus (HSV), hepatitis, and diabetes mellitus.

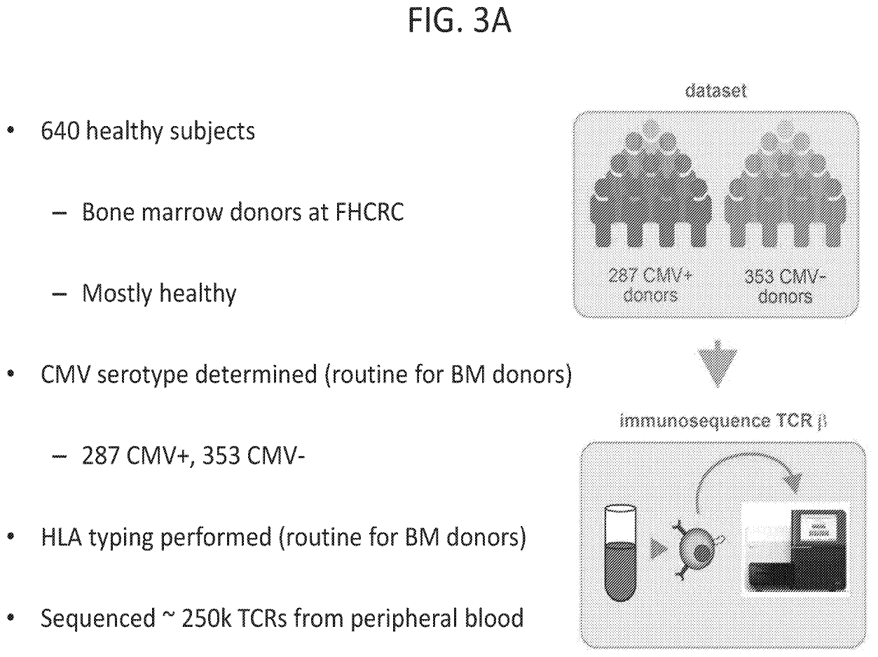

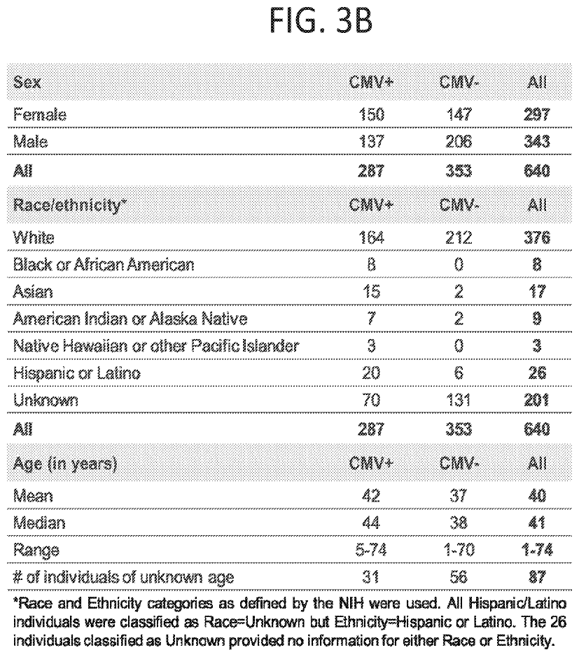

As shown in FIG. 3A, 640 subjects were specifically phenotyped for HLA type and CMV status for eligibility as a HCT donor, and segregated between 287 CMV+ donors and 353 CMV− donors. The CMV seropositive rate was approximately 45% (roughly equal numbers of seropositive and seronegative samples for the investigation of public T cell responses).

FIG. 3B shows demographic characteristics of the subjects included in this study, classified by CMV status.

### High-Throughput TCRβ Sequencing:

Genomic DNA was extracted from peripheral blood samples using the Qiagen DNeasy Blood extraction Kit (Qiagen, Gaithersburg, MD, USA). The CDR3 regions of rearranged TCRβ genes were sequenced; the TCRß CDR3 region was defined according to the IMGT collaboration^{28, 20}. TCRβ CDR3 regions were amplified and sequenced using methods described above and in previously described protocols^{5, 29}. The multiplexed PCR method used a mixture of 60 forward primers specific to TCR Vβ gene segments and 13 reverse primers specific to TCR Jβ gene segments. The resulting amplicons were sequenced using the methods described above. Reads of 87 bp were obtained using the Illumina HiSeq System. Raw HiSeq sequence data were preprocessed to remove errors in the primary sequence of each read, and to compress the data. A nearest neighbor algorithm was used to collapse the data into unique sequences by merging closely related sequences, to remove both PCR and sequencing errors.

In order to ensure adequate coverage of each T cell rearrangement, 8-10× sequence coverage, or ˜6-10 million sequencing reads per sample, was generated using approximately eight full sequencing runs. All sequencing reads were processed using a standardized bioinformatics pipeline to 1) demultiplex reads to specific samples, 2) eliminate low quality sequence and remove potential contaminants, 3) align and identify specific TCRß V and J gene segments and CDR3 regions, 4) cluster highly similar sequences to account for PCR and sequencing errors, 5) normalize data to remove PCR amplification bias, 6) estimate total T-cell input, and 7) generate TCRβ unique sequence counts and distributions.

Here, approximately 250,000 rearranged T cell receptor genes were sequenced from peripheral blood of each subject.

### Feature Selection:

Across 640 subjects, there were 185,204 (+/−84,171) unique TCRβs per subject, and 83,727,796 unique TCRβs in aggregate. Rather than attempting high dimensional CMV classification using all unique TCRβs as potential features, a novel feature selection scheme was developed. Feature selection was selection of a particular amino acid sequence to identify common TCRs among individuals with CMV.

Since many clones were unique to a single subject (and consequently unique to either the CMV+ or CMV− classes), it was vital to control false discovery rate in feature selection to avoid over-fitting to the many spurious associations of unique TCRβs with CMV status.

Each unique TCRβ rearrangement, identified by V and J gene assignment and CDR3 amino acid sequence, was tested for CMV association. Each of these was subjected to a one-tailed Fisher exact test for its incidence in CMV− and CMV+ subjects. Specifically, letting nᵢⱼ denote the number of subjects with CMV status j (with j− or +) and clone i present, a p-value pᵢ was computed by performing Fisher's exact test on the contingency table.

| | CMV+ | CMV− |
|---|---|---|
| TCRβ sequence i present | nᵢ₊ | nᵢ₋ |
| TCRβ sequence i not present | N₊ − nᵢ₊ | N₋ − nᵢ₋ |

where N₊ and N₋ denote the total number of subjects having each CMV status (CMV+ and CMV− respectively).

To characterize a rejection region in the presence of many weakly dependent hypotheses (one for each unique TCRB), CMV status assignments were randomly permuted 100 times, and statistics on the number of rejections at the nominal p-value threshold were calculated. Approximating the total fraction of true null hypotheses as unity, this allowed estimation of the false discovery rate (FDR) as the ratio of the mean number of rejections under permutation to the actual number of rejections.

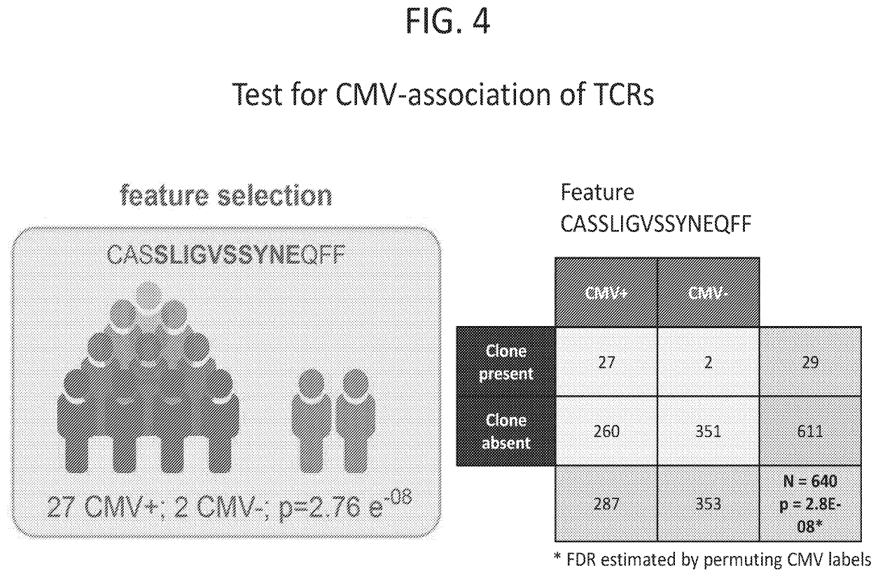

As shown by example in FIG. 4, the TCR amino acid sequence “CASSLIGVSSYNEQFF” (SEQ ID NO: 12) was selected and was identified in 27 CMV+ subjects and 2 CMV− subjects (p=2.8E-08).

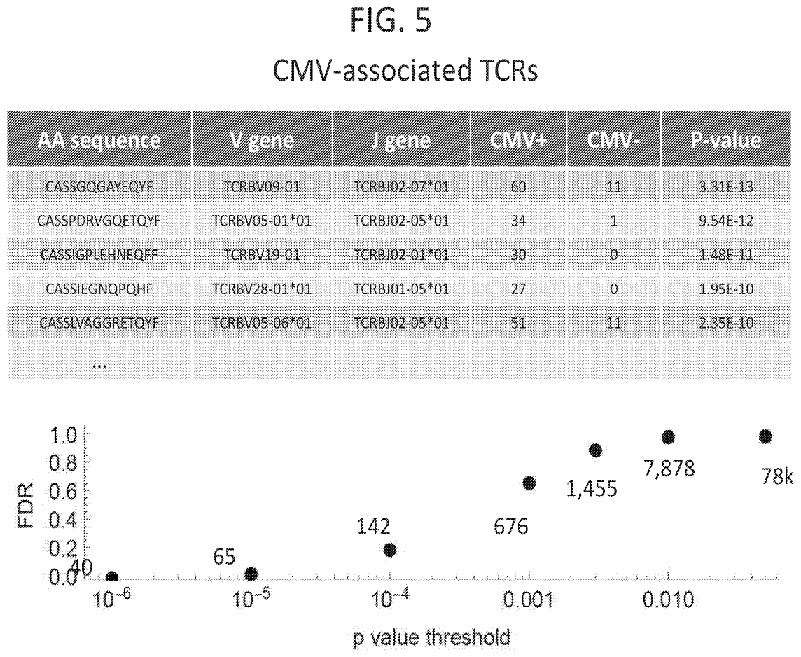

FIG. 5 shows exemplary TCR amino acid sequences identified in CMV+ subjects. As shown in Table 1, at a selected p value of 10⁻⁴, 142 public T cell clones were identified as associated with CMV+ subjects. The p value was chosen at 10⁻⁴, but as one can see, at a lower p value, larger numbers of CMV associated TCRß sequences can be identified.

Table 1: Each of the 142 TCRß sequences significantly associated with CMV status (p≤1×10⁻⁴, FDR˜20%) in the cohort was included. Sequences were defined using the amino acid sequence of the CDR3 region along with V and J gene segments. The number of CMV+ and CMV− subjects in which each sequence was observed is given, as well as the p-value from a Fisher exact test for association with CMV status, and any HLA-A or HLA-B alleles that were significantly associated (p≤1×10⁻³) with the presence of each sequence.

TABLE 1
Exemplary CMV-Associated TCRβ Sequences

| SEQ | | | | | | |
|---|---|---|---|---|---|---|
| ID | | | | CMV+/ | Fisher | HLA |
| NO: | AA sequence | V gene | J gene | CMV− | p value | association(s) |
| 1 | CASSGQGAYEQYF | TCRBV09-01 | TCRBJ02-07*03 | 60/11 | 3.3142E−13 | A29, B13 |
| 2 | CASSPDRVGQETQYF | TCRBV05-01*01 | TCRBJ02-05*03 | 34/1 | 9.5395E−12 | None |
| 3 | CASSIGPLEHNEQFF | TCRBV19-01 | TCRBJ02-01*03 | 30/0 | 1.4752E−11 | A1, B8 |
| 4 | CASSIEGNQPQHF | TCRBV28-01*01 | TCRBJ01-05*03 | 27/0 | 1.9463E−10 | None |
| 5 | CASSLVAGGRETQYF | TCRBV05-06*01 | TCRBJ02-05*03 | 51/11 | 2.3538E−10 | B8 |
| 6 | CASSLEAEYEQYF | TCRBV07-02*01 | TCRBJ02-07*03 | 30/1 | 2.7085E−10 | B8 |
| 7 | CATSDGDEQFF | TCRBV24 | TCRBJ02-01*03 | 41/6 | 4.7339E−10 | A1 |
| 8 | CASSLAPGATNEKLFF | TCRBV07-06*01 | TCRBJ01-04*03 | 36/4 | 9.134E−10 | A2 |
| 9 | CASSRGRQETQYF | TCRBV07-06*01 | TCRBJ02-05*03 | 37/5 | 2.1095E−09 | None |
| 10 | CASSAGQGVTYEQYF | TCRBV09-01 | TCRBJ02-07*03 | 23/0 | 5.8832E−09 | A1, B8 |
| 11 | CASSERREKLFF | TCRBV05-06*01 | TCRBJ01-04*03 | 28/2 | 1.2553E−08 | A29, B50 |
| 12 | CASSLIGVSSYNEQFF | TCRBV07-09 | TCRBJ02-01*03 | 27/2 | 2.7588E−08 | A3, B7 |
| 13 | CASSFPTSGQETQYF | TCRBV07-09 | TCRBJ02-05*03 | 27/2 | 2.7588E−08 | A24 |
| 14 | CASSPQRNTEAFF | TCRBV04-03*01 | TCRBJ01-01*03 | 27/2 | 2.7588E−08 | A3, B7 |
| 15 | CSVRDNFNQPQHF | TCRBV29-01*01 | TCRBJ01-05*03 | 21/0 | 3.1927E−08 | None |
| 16 | CATSRDSQGSYGYTF | TCRBV15-01*01 | TCRBJ01-02*03 | 21/0 | 3.1927E−08 | None |
| 17 | CASSPGDEQYF | TCRBV25-01*01 | TCRBJ02-07*03 | 24/1 | 3.7138E−08 | B7 |
| 18 | CASSQTGGRNQPQHF | TCRBV12 | TCRBJ01-05*03 | 26/2 | 6.0357E−08 | A2 |
| 19 | CASSQNRGQETQYF | TCRBV14-01*01 | TCRBJ02-05*03 | 26/2 | 6.0357E−08 | None |
| 20 | CASSLVIGGDTEAFF | TCRBV05-01*01 | TCRBJ01-01*03 | 26/2 | 6.0357E−08 | B8 |
| 21 | CASSFHGFNQPQHF | TCRBV05-06*01 | TCRBJ01-05*03 | 20/0 | 7.4139E−08 | A1, B3 |
| 22 | CASSRLAGGTDTQYF | TCRBV07-03*01 | TCRBJ02-03*03 | 50/16 | 7.5065E−08 | A1, B8 |
| 23 | CASSLPSGLTDTQYF | TCRBV28-01*01 | TCRBJ02-03*03 | 25/2 | 1.3144E−07 | None |
| 24 | CATSRDTQGSYGYTF | TCRBV15-01*01 | TCRBJ01-02*03 | 19/0 | 1.7179E−07 | None |
| 25 | CATSDGDTQYF | TCRBV24 | TCRBJ02-03*03 | 51/18 | 2.2203E−07 | A1, B8 |
| 26 | CASSLVASGRETQYF | TCRBV05-06*01 | TCRBJ02-05*03 | 24/2 | 2.8483E−07 | None |
| 27 | CASSIWGLDTEAFF | TCRBV19-01 | TCRBJ01-01*03 | 20/4 | 3.6207E−07 | None |
| 28 | CASSPGDEQFF | TCRBV25-01*01 | TCRBJ02-01*03 | 28/4 | 3.6207E−07 | B7 |
| 29 | CASSPSTGTEAFF | TCRBV05-06*01 | TCRBJ01-01*03 | 18/0 | 3.9723E−07 | None |
| 30 | CSVEEDEGIYGYTF | TCRBV29-01*01 | TCRBJ01-02*03 | 18/0 | 3.9723E−07 | None |
| 31 | CASSEIPNTEAFF | TCRBV06-04 | TCRBJ01-01*03 | 18/0 | 3.9723E−07 | A24 |
| 32 | CASSQVPGQGDNEQFF | TCRBV14-01*01 | TCRBJ02-01*03 | 21/1 | 4.1428E−07 | A1, B8 |
| 33 | CASSPAGLNTEAFF | TCRBV19-01 | TCRBJ01-01*03 | 21/1 | 4.1428E−07 | None |
| 34 | CASSLGLKGTQYF | TCRBV12 | TCRBJ02-05*03 | 21/1 | 4.1428E−07 | None |
| 35 | CASSGDRLYEQYF | TCRBV02-01*01 | TCRBJ02-07*03 | 23/2 | 6.1415E−07 | None |
| 36 | CSVRDNYNQPQHF | TCRBV29-01*01 | TCRBJ01-05*03 | 23/2 | 6.1415E−07 | None |
| 37 | CASSYGGLGSYEQYF | TCRBV06-05*01 | TCRBJ02-07*03 | 23/2 | 6.1415E−07 | None |
| 38 | CASNRDRGRYEQYF | TCRBV06-01*01 | TCRBJ02-07*03 | 20/1 | 9.1836E−07 | B45 |
| 39 | CASMGGASYEQYF | TCRBV27-01*01 | TCRBJ02-07*03 | 20/1 | 9.1836E−07 | A2 |
| 40 | CASSLGVGPYNEQFF | TCRBV07-02*01 | TCRBJ02-01*03 | 20/1 | 9.1836E−07 | B7 |
| 41 | CASSLGGAGDTQYF | TCRBV12 | TCRBJ02-03*03 | 44/15 | 1.1802E−06 | None |
| 42 | CATSRGTVSYEQYF | TCRBV15-01*01 | TCRBJ02-07*03 | 30/6 | 1.2303E−06 | None |
| 43 | CATSDGETQYF | TCRBV24 | TCRBJ02-05*03 | 70/36 | 1.3031E−06 | A1, B52 |
| 44 | CASSEARGGVEKLFF | TCRBV06-01*01 | TCRBJ01-04*03 | 22/2 | 1.3173E−06 | None |
| 45 | CASSLNRGQETQYF | TCRBV14-01*01 | TCRBJ02-05*03 | 19/1 | 2.0272E−06 | None |
| 46 | CSVRDNHNQPQHF | TCRBV29-01*01 | TCRBJ01-05*03 | 19/1 | 2.0272E−06 | None |
| 47 | CASSESGHRNQPQHF | TCRBV10-02*01 | TCRBJ01-05*03 | 16/0 | 2.1105E−06 | None |
| 48 | CSASPGQGASYGYTF | TCRBV20 | TCRBJ01-02*03 | 16/0 | 2.1105E−06 | None |
| 49 | CASSEARTRAFF | TCRBV06-01*01 | TCRBJ01-01*03 | 16/0 | 2.1105E−06 | None |
| 50 | CASRPTGYEQYF | TCRBV06-01*01 | TCRBJ02-07*03 | 21/2 | 2.8099E−06 | B39 |
| 51 | CASSVTGGTDTQYF | TCRBV09-01 | TCRBJ02-03*03 | 74/41 | 2.8214E−06 | A1, B8 |
| 52 | CASSRLAASTDTQYF | TCRBV07-03*01 | TCRBJ02-03*03 | 23/3 | 3.1389E−06 | A1, B8 |
| 53 | CATSDSVTNTGELFF | TCRBV24 | TCRBJ02-02*03 | 18/1 | 4.4551E−06 | B8 |
| 54 | CASSRNRESNQPQHF | TCRBV06-05*01 | TCRBJ01-05*03 | 18/1 | 4.4551E−06 | None |
| 55 | CASSAQGAYEQYF | TCRBV09-01 | TCRBJ02-07*03 | 2B/6 | 4.717E−06 | None |
| 56 | CASSIQGYSNQPQHF | TCRBV05-08*01 | TCRBJ01-05*03 | 15/0 | 4.8494E−06 | B8 |
| 57 | CASSYNPYSNQPQHF | TCRBV06-06 | TCRBJ01-05*03 | 15/0 | 4.8494E−06 | None |
| 58 | CASSLGHRDPNTGELFF | TCRBV05-01*01 | TCRBJ02-02*03 | 15/0 | 4.8494E−06 | B13 |
| 59 | CASSTTGGDGYTF | TCRBV19-01 | TCRBJ01-02*03 | 26/5 | 5.5727E−06 | None |
| 60 | CASSVLAGPTDTQYF | TCRBV09-01 | TCRBJ02-03*03 | 26/5 | 5.5727E−06 | A1 |
| 61 | CASSYRQETQYF | TCRBV06-05*01 | TCRBJ02-05*03 | 20/2 | 5.9592E−06 | None |
| 62 | CASSSGQVYGYTF | TCRBV05-06*01 | TCRBJ01-02*03 | 22/3 | 6.4736E−06 | A1, B8 |
| 63 | CASGRDTYEQYF | TCRBV02-01*01 | TCRBJ02-07*03 | 17/1 | 9.7453E−06 | None |
| 64 | CATSDSRTGGQETQYF | TCRBV24 | TCRBJ02-05*03 | 17/1 | 9.7453E−06 | B8 |
| 65 | CASSSPGRSGANVLTF | TCRBV28-01*01 | TCRBJ02-06*03 | 17/1 | 9.7453E−06 | None |
| 66 | CASSYGGEGYTF | TCRBV06-05*01 | TCRBJ01-02*03 | 36/12 | 1.059E−05 | None |
| 67 | CASSLAGVDYEQYF | TCRBV07-09 | TCRBJ02-07*03 | 25/5 | 1.0951E−05 | B8 |
| 68 | CASSLQGADTQYF | TCRBV07-08*01 | TCRBJ02-03*03 | 14/0 | 1.112E−05 | None |
| 69 | CASSLEAENEQFF | TCRBV07-02*01 | TCRBJ02-01*03 | 14/0 | 1.112E−05 | A11, B8 |
| 70 | CASSEAPSTSTDTQYF | TCRBV02-01*01 | TCRBJ02-03*03 | 14/0 | 1.112E−05 | None |
| 71 | CASSLEGQQPQHF | TCRBV28-01*01 | TCRBJ01-05*03 | 14/0 | 1.112E−05 | None |
| 72 | CASSLGHRDSSYEQYF | TCRBV05-01*01 | TCRBJ02-07*03 | 14/0 | 1.112E−05 | A29, B57 |
| 73 | CASSPSRNTEAFF | TCRBV04-03*01 | TCRBJ01-01*03 | 19/2 | 1.2561E−05 | A3, B7 |
| 74 | CSALGHSNQPQHF | TCRBV20 | TCRBJ01-05*03 | 19/2 | 1.2561E−05 | None |
| 75 | CASSHRDRNYEQYF | TCRBV07-09 | TCRBJ02-07*03 | 19/2 | 1.2561E−05 | A24 |
| 76 | CASSPPGQGSDTQYF | TCRBV18-01*01 | TCRBJ02-03*03 | 28/7 | 1.3913E−05 | None |
| 77 | CASSLQGYSNQPQHF | TCRBV05-08*01 | TCRBJ01-05*03 | 34/11 | 1.4711E−05 | B8 |
| 78 | CASSYVRTGGNYGYTF | TCRBV06-05*01 | TCRBJ01-02*03 | 31/9 | 1.5052E−05 | None |
| 79 | CASSRDRNYGYTF | TCRBV06-04 | TCRBJ01-02*03 | 29/8 | 2.0024E−05 | None |
| 80 | CASSTGTSGSYEQYF | TCRBV06-01*01 | TCRBJ02-07*03 | 16/1 | 2.1213E−05 | B54 |
| 81 | CASRSDSGANVLTF | TCRBV06-04 | TCRBJ02-06*03 | 16/1 | 2.1213E−05 | None |
| 82 | CATSRVAGETQYF | TCRBV15-01*01 | TCRBJ02-05*03 | 24/5 | 2.1345E−05 | B7 |
| 83 | CASSEEGIQPQHF | TCRBV02-01*01 | TCRBJ01-05*03 | 22/4 | 2.4814E−05 | None |
| 84 | CASSLGGPGDTQYF | TCRBV12 | TCRBJ02-03*03 | 22/4 | 2.4814E−05 | None |
| 85 | CASSLVAAGRETQYF | TCRBV05-06*01 | TCRBJ02-05*03 | 13/0 | 2.5446E−05 | B8 |
| 86 | CASRGQGWDEKLFF | TCRBV06-05*01 | TCRBJ01-04*03 | 13/0 | 2.5446E−05 | None |
| 87 | CASSLEGQGFGYTF | TCRBV05-01*01 | TCRBJ01-02*03 | 13/0 | 2.5446E−05 | None |
| 88 | CASRDWDYTDTQYF | TCRBV02-01*01 | TCRBJ02-03*03 | 13/0 | 2.5446E−05 | None |
| 89 | CASSRSGLAGNTGELFF | TCRBV06 | TCRBJ02-02*03 | 13/0 | 2.5446E−05 | None |
| 90 | CASSPGQEAGANVLTF | TCRBV05-01*01 | TCRBJ02-06*03 | 13/0 | 2.5446E−05 | None |
| 91 | CASSLGDRPDTQYF | TCRBV11-02*02 | TCRBJ02-03*03 | 13/0 | 2.5446E−05 | None |
| 92 | CASSFPGGETQYF | TCRBV11-01*01 | TCRBJ02-05*03 | 18/2 | 2.6306E−05 | None |
| 93 | CASSLETYGYTF | TCRBV05-06*01 | TCRBJ01-02*03 | 18/2 | 2.6306E−05 | B78 |
| 94 | CASSSGQVQETQYF | TCRBV11-02*02 | TCRBJ02-05*03 | 18/2 | 2.6306E−05 | B51 |
| 95 | CASSFDNYGYTF | TCRBV05-04*01 | TCRBJ01-02*03 | 18/2 | 2.6306E−05 | None |
| 96 | CASSEGARQPQHF | TCRBV10-02*01 | TCRBJ01-05*03 | 18/2 | 2.6306E−05 | None |
| 97 | CASSLTGGRNQPQHF | TCRBV12 | TCRBJ01-05*03 | 33/11 | 2.6324E−05 | A2 |
| 98 | CASSLLWDQPQHF | TCRBV05-05*01 | TCRBJ01-05*03 | 20/3 | 2.6954E−05 | None |
| 99 | CASSLFGTGGNTEAFF | TCRBV05-06*01 | TCRBJ01-01*03 | 20/3 | 2.6954E−05 | B44 |
| 100 | CASSISAGEAFF | TCRBV19-01 | TCRBJ01-01*03 | 20/3 | 2.6954E−05 | None |
| 101 | CASSPPSGLTDTQYF | TCRBV28-01*01 | TCRBJ02-03*03 | 20/3 | 2.6954E−05 | None |
| 102 | CASSPLSDTQYF | TCRBV07-09 | TCRBJ02-03*03 | 30/9 | 2.752E−05 | None |
| 103 | CASSSRGTGELFF | TCRBV28-01*01 | TCRBJ02-02*03 | 25/6 | 3.3358E−05 | None |
| 104 | CASSYAGDGYTF | TCRBV06-05*01 | TCRBJ01-02*03 | 31/10 | 3.6414E−05 | B8 |
| 105 | CASSDRGNTGELFF | TCRBV04-01*01 | TCRBJ02-02*03 | 31/10 | 3.6414E−05 | A68, B41 |
| 106 | CASSPGGTQYF | TCRBV12 | TCRBJ02-05*03 | 28/8 | 3.6921E−05 | None |
| 107 | CASSLGDRAYNEQFF | TCRBV05-06*01 | TCRBJ02-01*03 | 28/8 | 3.6921E−05 | None |
| 108 | CASSLRGSSYNEQFF | TCRBV05-08*01 | TCRBJ02-01*03 | 23/5 | 4.1254E−05 | None |
| 109 | CASSLTASSYEQYF | TCRBV05-01*01 | TCRBJ02-07*03 | 23/5 | 4.1254E−05 | None |
| 110 | CSASDHEQYF | TCRBV20-01*01 | TCRBJ02-07*03 | 23/5 | 4.1254E−05 | None |
| 111 | CASSQGRHTDTQYF | TCRBV14-01*01 | TCRBJ02-03*03 | 15/1 | 4.5934E−05 | A68 |
| 112 | CASSRPGQGNTEAFF | TCRBV12 | TCRBJ01-01*03 | 26/7 | 4.8714E−05 | None |
| 113 | CASSLVGDGYTF | TCRBV07-08*01 | TCRBJ01-02*03 | 36/14 | 4.9771E−05 | None |
| 114 | CASSLGAGNQPQHF | TCRBV28-01*01 | TCRBJ01-05*03 | 29/9 | 4.9861E−05 | None |
| 115 | CASSLTDTGELFF | TCRBV11-02*02 | TCRBJ02-02*03 | 29/9 | 4.9861E−05 | None |
| 116 | CASSLTGGNSGNTIYF | TCRBV07-02*01 | TCRBJ01-03*03 | 19/3 | 5.4373E−05 | B14 |
| 117 | CAWRGTGNSPLHF | TCRBV30-01*01 | TCRBJ01-06*03 | 17/2 | 5.4712E−05 | None |
| 118 | CASASANYGYTF | TCRBV12 | TCRBJ01-02*03 | 12/0 | 5.8109E−05 | A2 |
| 119 | CASSLQAGANEQFF | TCRBV07-02*01 | TCRBJ02-01*03 | 12/0 | 5.8109E−05 | None |
| 120 | CASSEEAGGSGYTF | TCRBV06-01*01 | TCRBJ01-02*03 | 12/0 | 5.8109E−05 | None |
| 121 | CASRTGESGYTF | TCRBV06-05*01 | TCRBJ01-02*03 | 12/0 | 5.8109E−05 | None |
| 122 | CASSGLNEQFF | TCRBV06-01*01 | TCRBJ02-01*03 | 12/0 | 5.8109E−05 | None |
| 123 | CASSWDRDNSPLHF | TCRBV25-01*01 | TCRBJ01-06*03 | 12/0 | 5.8109E−05 | B7 |
| 124 | CASSIRTNYYGYTF | TCRBV19-01 | TCRBJ01-02*03 | 12/0 | 5.8109E−05 | None |
| 125 | CSARSPEAFF | TCRBV20-01*01 | TCRBJ01-01*03 | 12/0 | 5.8109E−05 | None |
| 126 | CASSRGTGATDTQYF | TCRBV19-01 | TCRBJ02-03*03 | 12/0 | 5.8109E−05 | None |
| 127 | CASSPRVSNQPQHF | TCRBV12 | TCRBJ01-05*03 | 12/0 | 5.8109E−05 | None |
| 128 | CAISESQDRGHEQYF | TCRBV10-03*01 | TCRBJ02-07*03 | 12/0 | 5.8109E−05 | None |
| 129 | CASSLGRGYEKLFF | TCRBV05-06*01 | TCRBJ01-04*03 | 12/0 | 5.8109E−05 | B62 |
| 130 | CSVEVRGTDTQYF | TCRBV29-01*01 | TCRBJ02-03*03 | 12/0 | 5.8109E−05 | None |
| 131 | CASRGQGAGELFF | TCRBV02-01*01 | TCRBJ02-02*03 | 30/10 | 6.4924E−05 | None |
| 132 | CATSREGSGYEQYF | TCRBV15-01*01 | TCRBJ02-07*03 | 22/5 | 7.9023E−05 | None |
| 133 | CASSLGWTEAFF | TCRBV05-01*01 | TCRBJ01-01*03 | 22/5 | 7.9023E−05 | None |
| 134 | CASSLGSSSYNEQFF | TCRBV07-02*01 | TCRBJ02-01*03 | 35/14 | 8.4988E−05 | None |
| 135 | CASSSAGADTQYF | TCRBV07-09 | TCRBJ02-03*03 | 14/1 | 9.8907E−05 | B5 |
| 136 | CASSERKNYGYTF | TCRBV06-01*01 | TCRBJ01-02*03 | 14/1 | 9.8907E−05 | None |
| 137 | CASRDRDRVNTEAFF | TCRBV06-01*01 | TCRBJ01-01*03 | 14/1 | 9.8907E−05 | A3 |
| 138 | CASSRVGEQFF | TCRBV03 | TCRBJ02-01*03 | 14/1 | 9.8907E−05 | None |
| 139 | CASSPRWQETQYF | TCRBV27-01*01 | TCRBJ02-05*03 | 14/1 | 9.8907E−05 | B14 |
| 140 | CASTPGDTIYF | TCRBV25-01*01 | TCRBJ01-03*03 | 14/1 | 9.0907E−05 | B55 |
| 141 | CASSENGGNQPQHF | TCRBV06-01*01 | TCRBJ01-05*03 | 14/1 | 9.8907E−05 | None |
| 142 | CASSYPGETQYF | TCRBV06-06 | TCRBJ02-05*03 | 36/15 | 9.991E−05 | None |

### Dimensionality Reduction and Machine Learning:

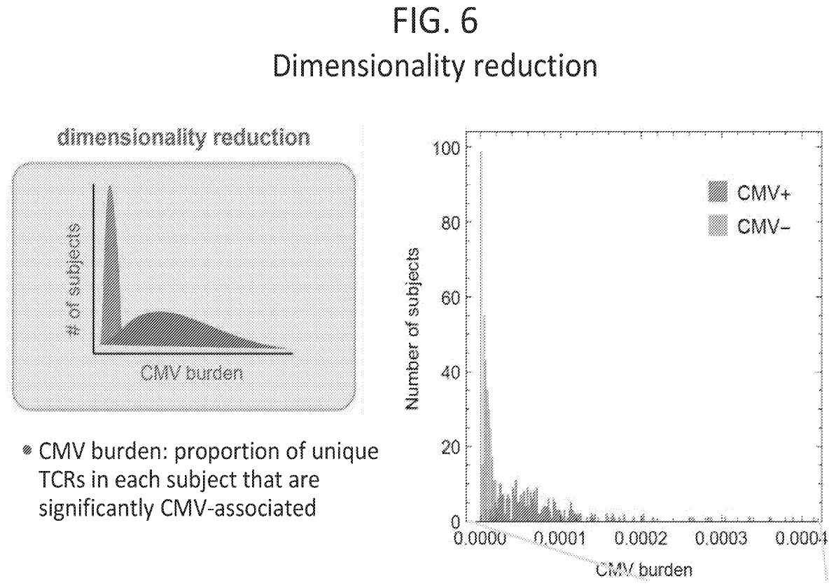

CMV burden was calculated for each subject. CMV burden is defined as the fraction of a subject's unique TCRβs that are significantly CMV-associated, as shown in FIG. 6. The graph in FIG. 6 shows the distribution of CMV scores (i.e., the proportion of each subject's TCRß repertoire that matches our list of 142 CMV-associated TCRβ sequences) among CMV+ and CMV− subjects.

This enabled fast training of a one-dimensional logistic regression classifier of CMV status. Exhaustive leave-one-out cross validation (including re-computation of CMV-associated clones) was performed, indicating high accuracy across a broad range of p-value thresholds, with performance degrading at a high FDR.

Each subject was removed from the dataset in turn, and the list of significantly CMV-associated TCRβs was re-calculated using the remaining subjects with known CMV status. Each subject's CMV burden was then calculated as described above. A one-dimensional logistic regression classifier was then trained on CMV burden vs. CMV status, and the CMV burden of the subject which was held out was then calculated and input into the logistic regression. The output of this logistic regression model was the probability that the subject of unknown status was CMV+, which was then compared to the (known but not leveraged) status of the held-out subject to determine the accuracy of the hold-out classification. Classification of a subject with genuinely unknown CMV status proceeds likewise: sequence TCRB, calculate a CMV burden (as proportion of unique TCRβs from this subject present on the list of CMV-associated TCRβs), and input this CMV burden into a logistic regression model trained on subjects of known status, with an estimated probability of CMV-positivity as output.

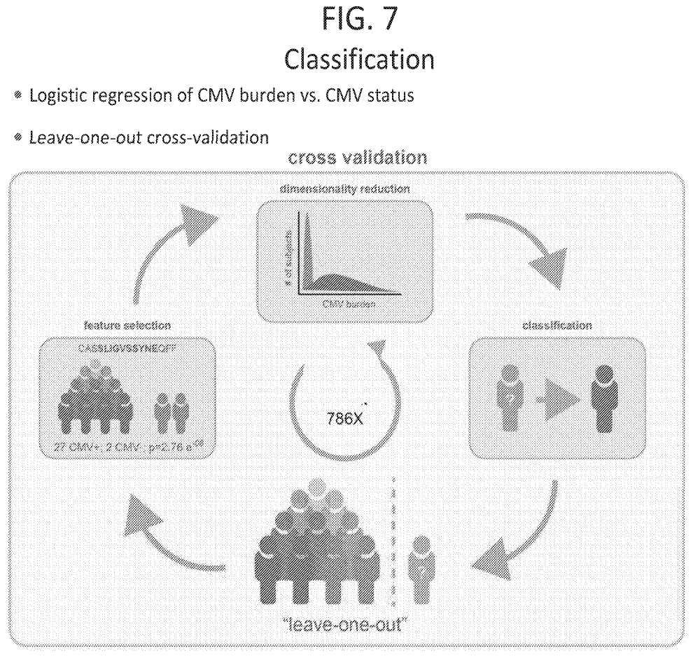

As shown in FIG. 7, the cross-validation method left out one of the initial 640 samples, then the database of CMV-specific TCRβ sequences and associated statistics was retrained in an unsupervised fashion to eliminate bias, and the CMV serostatus of the left-out sample was classified.

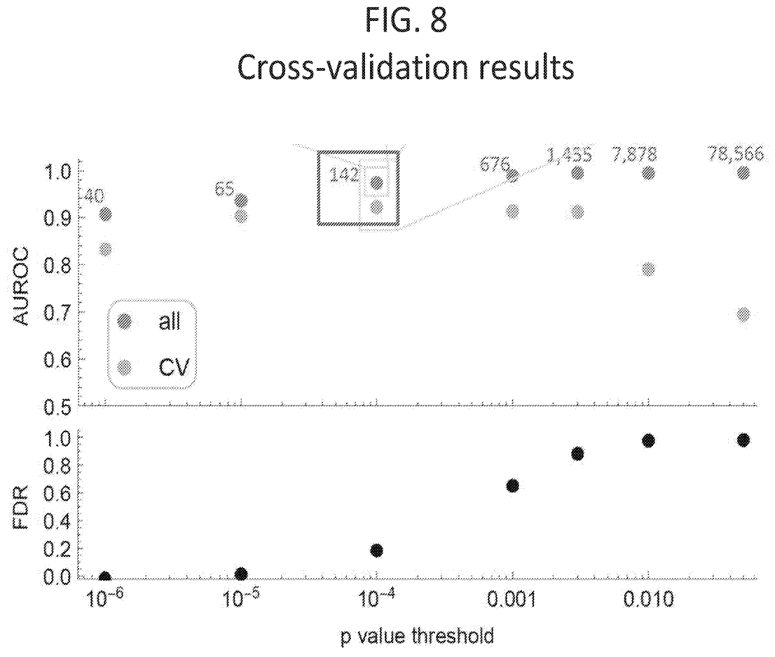

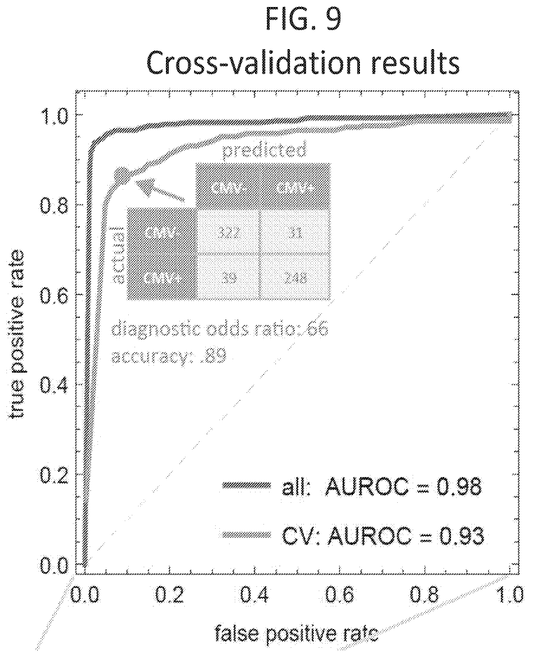

The cross-validated results are shown in FIGS. 8 and 9 (shown for all subjects and cross-validated (CV) subjects). The accuracy of the model was shown to be at 89%, corresponding to a diagnostic odds ratio of ˜66. Assessing model performance across the range of specificity vs. sensitivity using an ROC curve, an area under the ROC curve of 93% was achieved.

FIG. 8 (top graph) shows data for the classification performance of all and the cross-validation (CV) datasets for each p-value threshold, measured as the area under the ROC curve (AUROC). The number above each set of data points corresponds to the number of CMV-associated TCRβ identified at that p-value threshold, and the rectangle indicates the dataset selected for downstream analysis (p-value=10⁻⁴).

FIG. 8 (bottom graph) also shows a false discovery rate (FDR) estimated for each p-value threshold used in the identification of significantly CMV-associated TCRβ sequences, using permutations of CMV status. The best performance is seen at a p-value of 10⁻⁴, which corresponds to an estimated FDR of ˜20%, resulting in the identification of a set of 142 TCRβ sequences that were significantly associated with positive CMV status (listed in Table 1). Using these conditions resulted in a good separation between the CMV+ and CMV− subjects in the cohort as measured by CMV score (See FIG. 6).

FIG. 9 shows the ROC curves for both the all and the cross-validation datasets. The AUROC for the full dataset is 0.98, indicating that our approach resulted in an excellent classifier for CMV status. At the point of highest discriminating power, an accuracy of 0.89 and a diagnostic odds ratio of 66 in the cross-validation dataset was observed (achieved when classifying 86% of true positives correctly with a false positive rate of 8%). Taken together, these data suggest that that presence of public T cell responses to CMV is highly correlated with CMV positive status.

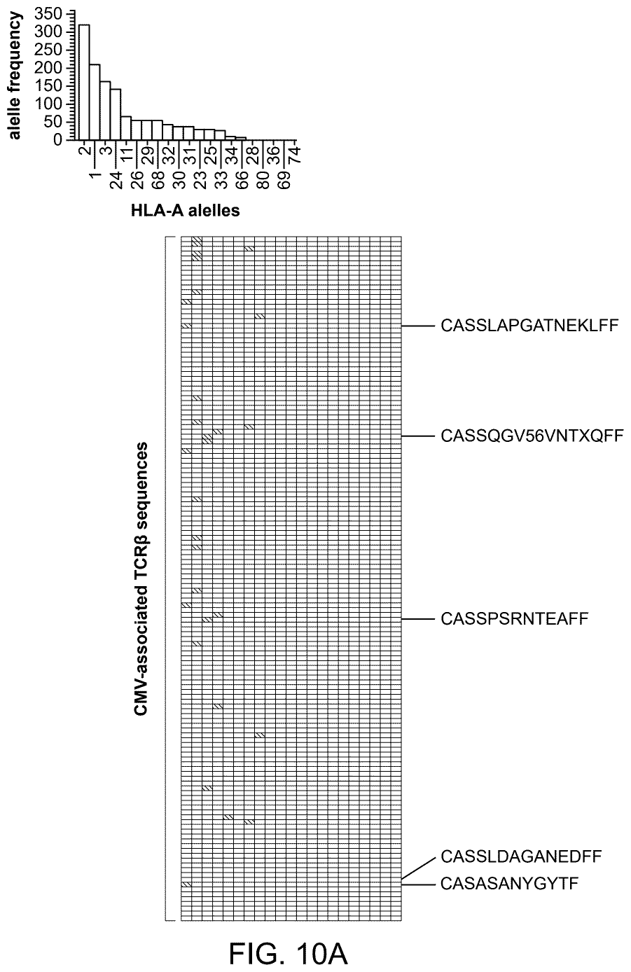

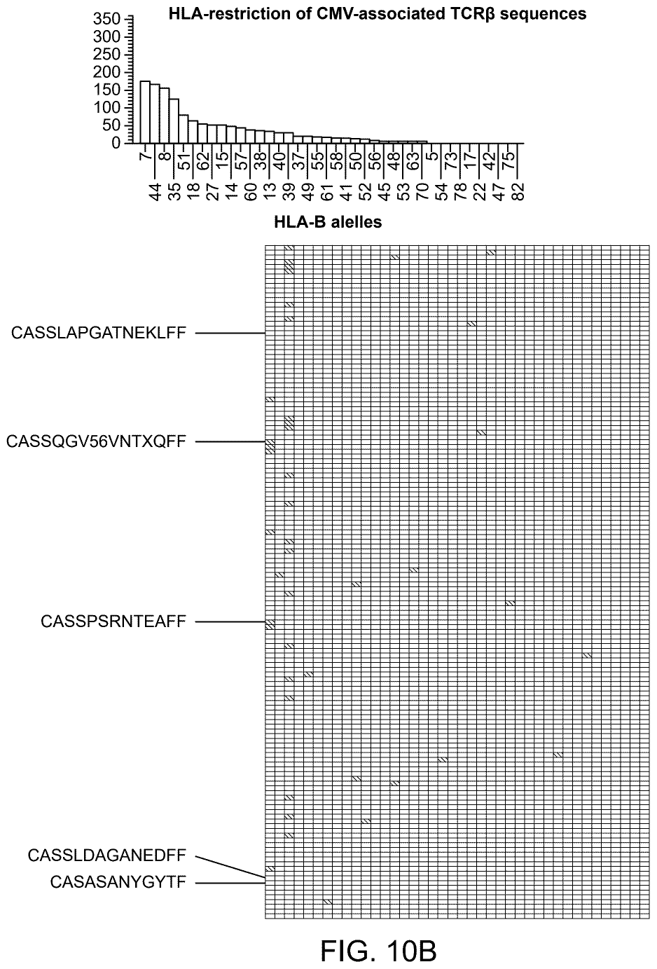

Given that T cells recognize their cognate antigens in the context of MHC molecules expressed by antigen presenting cells, it was tested whether the HLA-restriction of our CMV-associated TCRβ sequences could be identified. A Fisher's exact test was performed on each CMV-associated TCRβ sequence to determine if its presence was significantly associated with any of the HLA alleles observed in the cohort. The association of 57 out of 142 CMV-associated TCRβ sequences could be confidently assigned with at least one HLA allele, with a p-value cutoff of 1×10⁻³. Full results are presented in Table 1 (above) and FIG. 10.

FIGS. 10A and 10B show HLA-restriction of CMV-associated TCRβ sequences. FIG. 10A shows the distribution of HLA-A alleles in this cohort. FIG. 10B shows the distribution of HLA-B alleles in this cohort. Each of the 142 CMV-associated TCRβ sequences identified at p≤1×10⁻⁴ was tested for significant association with each HLA allele, with a p-value threshold of 1×10⁻³. Of these 142 CMV-associated TCRβ sequences, 57 were significantly associated with an HLA-A and/or an HLA-B allele, and no sequences were significantly associated with more than a single allele from each locus. Colored boxes and amino acid sequences indicate the 5 TCRβ sequences identified in our study that had been previously identified. In 4 of the cases, the correct HLA association was recapitulated, and in the 5^{th} case, there was no statistically-significant HLA association.

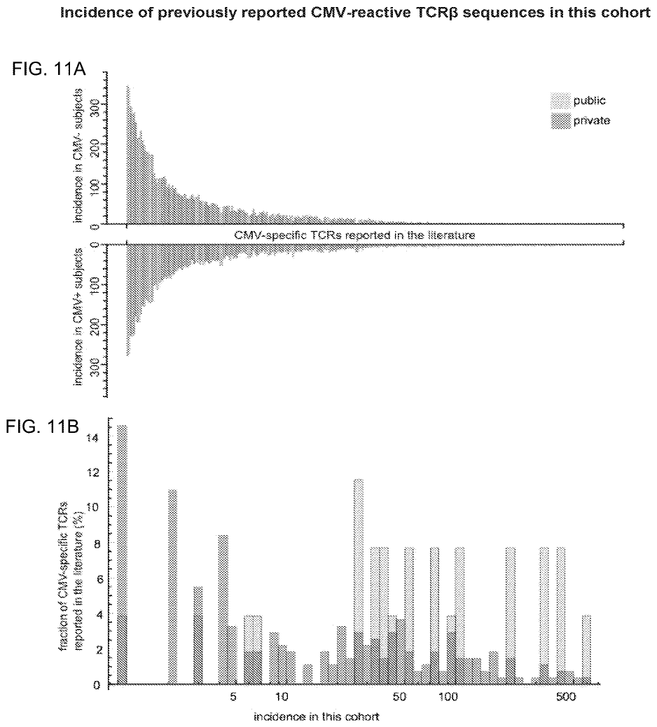

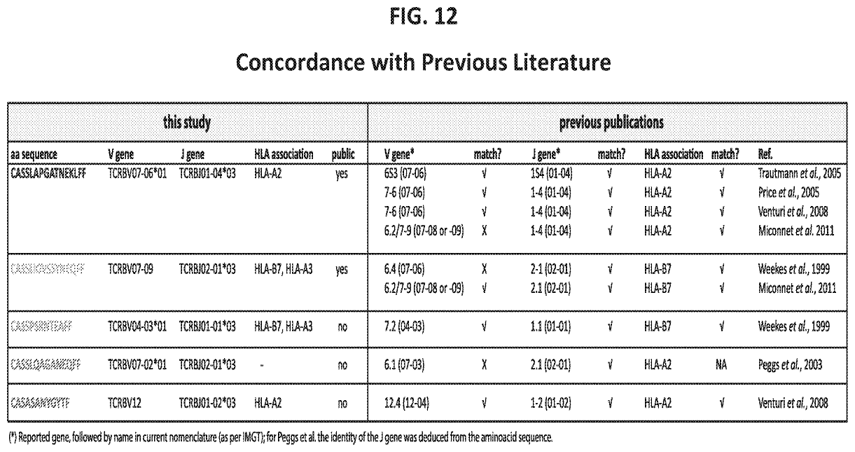

A literature search was performed to identify previously reported CMV-reactive TCRβ sequences, and 595 unique TCRβ sequences were selected that had been identified by at least one previously published study^{3, 10, 12-27}. Of these, 30 unique TCRβ sequences had previously been previously classified as public, or were reported in multiple studies. It was determined that 5 of these 30 public TCRβ sequences unique were contained in the set of 142 CMV-associated TCRβ sequences. Furthermore, an HLA association was identified for 4 of these 5 TCRβ sequences, and in all 4 cases our HLA association agreed with the literature. FIGS. 11A, 11B, and 12 provide more complete information on the prevalence in our dataset of previously identified CMV-reactive TCRβ sequences, as described below.

FIGS. 11A and 11B show the incidence of previously reported CMV-reactive TCRβ sequences in this cohort. After a literature search, 565 ‘private’ CMV-reactive TCRβ sequences (reported in one individual from a single study) and 30 ‘public’ CMV-reactive TCRβ sequences (reported in multiple studies or in multiple individuals within a single study) were identified. FIG. 11A shows the incidence of each such TCRβ sequence in the cohort of 640 subjects plotted along the horizontal axis by decreasing total incidence, with the incidence in CMV+ subjects above the horizontal and the incidence in CMV− subjects below the horizontal. Many previously-reported sequences were observed in the dataset, but most were seen in roughly equal number of CMV+ and CMV− subjects, which could be explained by receptor sequences with exceptionally high frequency in the naïve repertoire, or could reflect cross-reactive receptors that bind to CMV antigens but also other common antigens. FIG. 11B shows a histogram of incidence of these TCRβ sequences in the cohort of 640 subjects plotted for each group of sequences. Most previously-reported CMV-reactive TCRβ sequences were found in our dataset at appreciable levels, though only a handful were found disproportionately in CMV+ subjects at p≤1×10⁻⁴. ‘Public’ TRβ sequences reported in the literature were considerably more common in our cohort.

FIG. 12 shows the concordance of TCRB sequences in the cohort as compared to those in the literature. Of the 142 TCRβ sequences significantly associated with CMV status in the cohort with a p-value of less than 1×10⁻⁴, five TCRβ sequences (defined by matching CDR3 amino acid sequence) have been previously reported in the literature as public clones. FIG. 12 provides the CDR3 amino acid sequence, V and J genes, and HLA association for each sequence and compares it to previous reports. As expected, the two sequences previously identified as public were seen in more subjects in our cohort than the three sequences previously reported only once. Concordance of V gene, J gene, and HLA allele association with those reported in the literature was very good. Five out of nine total comparisons had the same V gene, and the other 4 comparisons had the same V gene subfamily. There were concordant J genes in 9 out of 9 comparisons, and an identical HLA association in 8 out of 9 comparisons, with one sequence not significantly HLA-associated in this study.

Thus, immunosequencing can be used to determine CMV-specific T cell sequences in a large cohort and predict CMV status in new subjects with high accuracy. In summary, it has been demonstrated that information gleaned from rearranged T cell receptors can be used to infer disease status based on the presence of public T cell responses; the only requirement is a large sample of pathogen-positive and -negative samples with which to identify these public T cell responses. Because high-throughput sequencing of T cell receptors captures all T cell responses equally, and these store immunological memory to all pathogens in a common format, reading T cell memory by looking for known public responses can be a viable strategy for simultaneously diagnosing a wide range of infectious agents using a single peripheral blood sample and a simple, unified assay. These methods can be applied needed to acute infections, given that T cell memory persists for years, accounting for the fact that it is not known how public clones decay with time after an acute infection. Accordingly, this method can be used to assess multiple infections simultaneously, such as HPV, EBV, CMV and others. The method can also be used to predict or diagnose a non-infectious (e.g. autoimmune) disease.

The methods of the invention can also be used to quantify disease burden (e.g. CMV reactivation post-transplant).

### Example 2: Identification of HLA Type

The first aim of the study was to create a comprehensive catalog of T cell receptor sequences and associated HLA types.

### HLA Typing:

640 subjects were phenotyped for HLA type according to standard protocol by the Fred Hutchinson Cancer Research Center.

### Immune Repertoire Sequencing:

Next, the TCRB sequences for each subject was determined by amplification and sequencing.

Genomic DNA was extracted from peripheral blood samples using the Qiagen DNeasy Blood extraction Kit (Qiagen, Gaithersburg, MD, USA). The CDR3 regions of rearranged TCRβ genes were sequenced; the TCRβ CDR3 region was defined according to the IMGT collaboration^{28, 20}. TCRβ CDR3 regions were amplified and sequenced using methods described above and in previously described protocols^{5, 29}. The multiplexed PCR method used a mixture of 60 forward primers specific to TCR Vβ gene segments and 13 reverse primers specific to TCR Jβ gene segments. The resulting amplicons were sequenced using the methods described above. Reads of 87 bp were obtained using the Illumina HiSeq System. Raw HiSeq sequence data were preprocessed to remove errors in the primary sequence of each read, and to compress the data. A nearest neighbor algorithm was used to collapse the data into unique sequences by merging closely related sequences, to remove both PCR and sequencing errors.

In order to ensure adequate coverage of each T cell rearrangement, 8-10× sequence coverage, or ˜6-10 million sequencing reads per sample, was generated using approximately eight full sequencing runs. All sequencing reads were processed using a standardized bioinformatics pipeline to 1) demultiplex reads to specific samples, 2) eliminate low quality sequence and remove potential contaminants, 3) align and identify specific TCRß V and J gene segments and CDR3 regions, 4) cluster highly similar sequences to account for PCR and sequencing errors, 5) normalize data to remove PCR amplification bias, 6) estimate total T-cell input, and 7) generate TCRβ unique sequence counts and distributions.

Here, approximately 250,000 rearranged T cell receptor genes were sequenced from peripheral blood of each subject. For each subject, approximately 10⁵ TCRB sequences were obtained, and the abundances of each unique sequence were quantified.

### Feature Selection: To Define Association Between an HLA Allele and TCRB Sequence(s)

For each subject, the total number of unique TCRB sequences and the frequency of each unique TCRB sequence are determined. For a unique TCRB sequence, it is determined how many subjects who are positive for an HLA allele have the TCRB sequence and how many subjects who are negative for the HLA allele have the TCRB sequence. In addition, it can be determined the number of subjects who are positive for an HLA allele and negative for the TCRB sequence and the number of subjects who are negative for the HLA allele and positive for the TCRB sequence. The table below shows categorization of subjects by the presence or absence of a TCRB sequence and the presence or absence of an HLA allele, HLA-A2.

| | HLA-A2+ | HLA-A2− |
|---|---|---|
| TCRβ sequence i present | nᵢ₊ | nᵢ₋ |
| TCRβ sequence i not present | N₊ − nᵢ₊ | N₋ − nᵢ₋ |

FIG. 14 shows a list of exemplary unique TCRB sequences and the number of subjects who are positive or negative for an HLA-A2 allele that have a particular TCRB sequence.

A p-value is determined for the association of each TCRB sequence with an HLA status using a Fisher exact test (two-tailed). The p value for association of each TCR with allele status using a Fisher exact test (two tailed) is calculated as follows:

$$Pr ( table ) = \frac{( \begin{matrix}{n}_{+} + {n}_{-} \\ {n}_{+}\end{matrix} ) ( \begin{matrix}{N}_{+} + {N}_{-} - {n}_{+} - {n}_{-} \\ {n}_{+} + {n}_{-}\end{matrix} )}{( \begin{matrix}{N}_{+} + {N}_{-} \\ {N}_{+}\end{matrix} )}$$

FIG. 14 shows exemplary p values that were calculated for the association of a particular TCRB sequence with an HLA type (HLA-A2).

As shown in FIG. 15, a p value is selected as a cutoff for identifying a set of “Feature TCRs” from the entire list of possible TCR sequences. Defining a p-value threshold and permuting the allele status across individuals provides an estimate of false discovery rate. This is performed for each HLA allele, resulting in a set of allele-associated TCRβ sequences for each HLA allele. In FIG. 15, a p value cutoff of p≤10⁻⁴ and an FDR of 0.1 is used to identify 288 TCRB sequences that are positively associated with HLA-A2. For each of the allele-associated TCRB sequences, the frequency of the sequence is also determined in each subject.

As shown in FIG. 15, a p value is selected as a cutoff for identifying a set of “Feature TCRS” from the entire list of possible TCR sequences. Defining a p-value threshold and permuting the allele status across individuals provides an estimate of false discovery rate. This is performed for each HLA allele, resulting in a set of allele-associated TCRβ sequences for each HLA allele. In FIG. 15, a p value cutoff of p≤10⁻⁴ and an FDR of 0.1 is used to identify 288 TCRB sequences that are positively associated with HLA-A2. For each of the allele-associated TCRB sequences, the frequency of the sequence is also determined in each subject.

The false discovery rate (FDR) was determined by permutation of allele status. See Storey et al. Statistical significance for genomewide studies. PNAS, 100 (6), pp. 9440-9445.

The feature selection step is followed by a machine learning process. As shown in FIG. 16, for each HLA allele, a logistic regression model is trained using the set of feature vectors over all subjects, along with the known status for presence of that allele.

As shown in FIG. 17, an exhaustive leave-one-out cross validation is performed where one subject is removed from the analysis, and the HLA status of the subject is inferred based on feature selection and training from only the remaining subjects. The result is a set of classifiers (one for each HLA allele) that estimate the probability of positive status for each HLA allele, taking as input the feature vector for each allele.

Equipped with these classifiers, the HLA type of a new subject can be assessed by: 1. immune repertoire sequencing, 2. computing feature vectors for each allele, and 3. defining a probability threshold for positive status that will be applied to the output from each classifier. In this manner, an HLA status can be inferred for a new subject with an unknown HLA status.

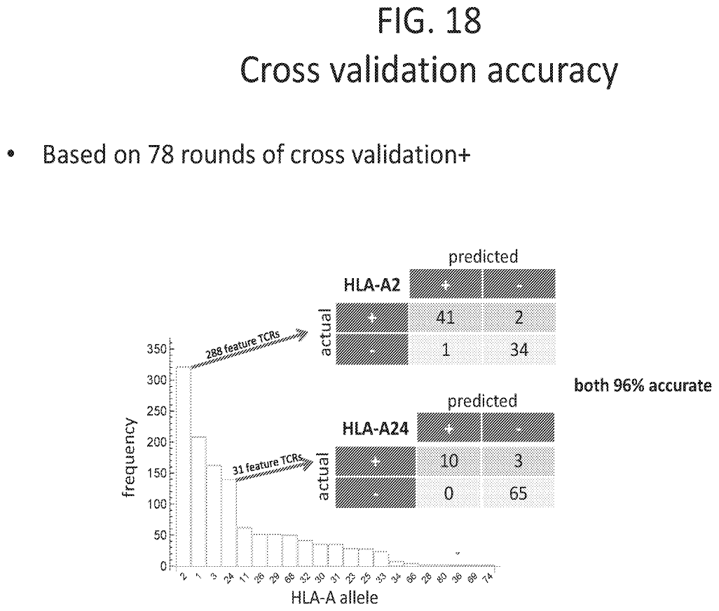

FIG. 18 shows the results of a cross validation study. The leave-one out cross validation was performed for 78 rounds. For the HLA-A2 allele, the method described above resulted in identification of 288 feature TCRs and an accurate prediction of 41 subjects out of 43 as HLA-A2 positive. As described above, the HLA type of the subjects was known prior to performing the cross validation. Only 2/43 subjects were false-positives for HLA-A2. Thus, the method was 96% accurate in predicting HLA-A2 presence in a subject based on the subject's TCRB sequence profile.

FIG. 18 also shows the results for cross-validation of the HLA-A24 allele, which also had a 96% accuracy. 10 out of 13 subjects were accurately predicted to possess the HLA-A24 allele, and 65 out of 65 subjects were accurately predicted to as not have the HLA-A24 allele based on the identified TCRB features.

### Example 3: HLA Study

In another example, the public T-cell response to cytomegalovirus (CMV) was investigated by sequencing rearranged T cell receptors (TCRs) in 650 subjects (294 with and 356 without CMV). The concordance between ˜90 million unique TCRs and CMV serostatus was assessed, focusing on identification of significant associations. In this study, 157 CMV-associated TCRs were identified at p≤10⁻³, FDR≈0.15) Training a binary classifier on these features, it was predicted that CMV serostatus in a leave-one-out cross-validation procedure had a diagnostic odds ratio of 44. The classifier was also tested on a second independent cohort of 120 subjects with known CMV serostatus, yielding a diagnostic odds ratio of 49.

Next, the HLA-restriction of each CMV-associated TCR was investigated by assessing the over-representation of particular HLA types among the subjects that carry each CMV-associated TCR. Of 157 CMV-associated TCRs, 61 were HLA-associated at p≤10⁻³. None of these were significantly associated with multiple HLA-A or HLA-B alleles.

There was substantial concordance between our data and previously published CMV- and HLA-associated TCRs. Most previously-reported public CMV-specific TCRs were seen in the data, although only 5/157 CMV-associated TCRs identified in this study have been previously reported. Of these, 4 were significantly HLA-restricted, and all four confirmed previous findings.

In addition, the association of TCRs with each HLA-A allele present in the cohort was investigated, with significant (p≤10⁻⁴) results for many higher frequency alleles. Binary classifier training resulted in high accuracy (˜96%) prediction for these alleles, indicating that HLA type can be inferred from immunosequencing data.

In summary, this study demonstrated the validity of association studies using immunosequencing for detection and HLA-association of public T-cell responses to infection, and showed that assessing the presence of associated T-cell responses can serve as a powerful diagnostic classifier.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

1. Neller, M. A., Burrows, J. M., Rist, M. J., Miles, J. J. & Burrows, S. R. High frequency of herpesvirus-specific clonotypes in the human T cell repertoire can remain stable over decades with minimal turnover. J Virol 87, 697-700 (2013).
2. Li, H., Ye, C., Ji, G. & Han, J. Determinants of public T cell responses. Cell Res 22, 33-42 (2012).
3. Venturi, V., Price, D. A., Douck, D. C. & Davenport, M. P. The molecular basis for public T-cell responses? Nat Rev Immunol 8, 231-238 (2008).
4. Arstila, T. P. et al. A direct estimate of the human alphabeta T cell receptor diversity. Science 286, 958-961 (1999).
5. Robins, H. S. et al. Comprehensive assessment of T-cell receptor beta-chain diversity in alphabeta T cells. Blood 114, 4099-4107 (2009).
6. Cabaniols, J. P., Fazilleau, N., Casrouge, A., Kourilsky, P. & Kanellopoulos, J. M. Most alpha/beta T cell receptor diversity is due to terminal deoxynucleotidyl transferase. J Exp Med 194, 1385-1390 (2001).
7. Davis, M. M. & Bjorkman, P. J. T-cell antigen receptor genes and T-cell recognition. Nature 334 (1988).
8. Robins, H. S. et al. Overlap and effective size of the human CD8+ T cell receptor repertoire. Sci Transl Med 2, 47ra64 (2010).
9. Li, H. et al. Recombinatorial biases and convergent recombination determine interindividual TCRbeta sharing in murine thymocytes. J Immunol 189, 2404-2413 (2012).
10. Venturi, V. et al. TCR beta-chain sharing in human CD8+ T cell responses to cytomegalovirus and EBV. J Immunol 181, 7853-7862 (2008).
11. Gandhi, M. K. & Khanna, R. Human cytomegalovirus: clinical aspects, immune regulation, and emerging treatments. Lancet Infect Dis 4, 725-738 (2004).
12. Babel, N. et al. Clonotype analysis of cytomegalovirus-specific cytotoxic T lymphocytes. J Am Soc Nephrol 20, 344-352 (2009).
13. Iancu, E. M. et al. Clonotype selection and composition of human CD8 T cells specific for persistent herpes viruses varies with differentiation but is stable over time. J Immunol 183, 319-331 (2009).
14. Khan, N., Cobbold, M., Keenan, R. & Moss, P. A. Comparative analysis of CD8+ T cell responses against human cytomegalovirus proteins pp65 and immediate early 1 shows similarities in precursor frequency, oligoclonality, and phenotype. J Infect Dis 185, 1025-1034 (2002).
15. Klarenbeek, P. L. et al. Deep sequencing of antiviral T-cell responses to HCMV and EBV in humans reveals a stable repertoire that is maintained for many years. PLOS Pathog 8, e1002889 (2012).
16. Klinger, M. et al. Combining next-generation sequencing and immune assays: a novel method for identification of antigen-specific T cells. PLOS One 8, e74231 (2013).
17. Koning, D. et al. In vitro expansion of antigen-specific CD8(+) T cells distorts the T-cell repertoire. J Immunol Methods 405, 199-203 (2014).
18. Miconnet, I. et al. Large TCR diversity of virus-specific CD8 T cells provides the mechanistic basis for massive TCR renewal after antigen exposure. J Immunol 186, 7039-7049 (2011).
19. Price, D. A. et al. Avidity for antigen shapes clonal dominance in CD8+ T cell populations specific for persistent DNA viruses. J Exp Med 202, 1349-1361 (2005).
20. Retiere, C. et al. Generation of cytomegalovirus-specific human T-lymphocyte clones by using autologous B-lymphoblastoid cells with stable expression of pp65 or IE1 proteins: a tool to study the fine specificity of the antiviral response. J Virol 74, 3948-3952 (2000).
21. Scheinberg, P. et al. The transfer of adaptive immunity to CMV during hematopoietic stem cell transplantation is dependent on the specificity and phenotype of CMV-specific T cells in the donor. Blood 114, 5071-5080 (2009).
22. Schub, A., Schuster, I. G., Hammerschmidt, W. & Moosmann, A. CMV-specific TCR-22 transgenic T cells for immunotherapy. J Immunol 183, 6819-6830 (2009).
23. Trautmann, L. et al. Selection of T cell clones expressing high-affinity public TCRs within Human cytomegalovirus-specific CD8 T cell responses. J Immunol 175, 6123-6132 (2005).
24 Wynn, K. K. et al. Impact of clonal competition for peptide-MHC complexes on the CD8+ T-cell repertoire selection in a persistent viral infection. Blood 111, 4283-4292 (2008).
25. Peggs, K. et al. Characterization of human cytomegalovirus peptide-specific CD8(+) T-cell repertoire diversity following in vitro restimulation by antigen-pulsed dendritic cells. Blood 99, 213-223 (2002).
26. Weekes, M. P., Wills, M. R., Mynard, K., Carmichael, A. J. & Sissons, J. G. The memory cytotoxic T-lymphocyte (CTL) response to human cytomegalovirus infection contains individual peptide-specific CTL clones that have undergone extensive expansion in vivo. J Virol 73, 2099-2108 (1999).
27. Lim, A. et al. Frequent contribution of T cell clonotypes with public TCR features to the chronic response against a dominant EBV-derived epitope: application to direct detection of their molecular imprint on the human peripheral T cell repertoire. J Immunol 165, 2001-2011 (2000).
28. Yousfi Monod, M., Giudicelli, V., Chaume, D. & Lefranc, M. P. IMGT/JunctionAnalysis: the first tool for the analysis of the immunoglobulin and T cell receptor complex V-J and V-D-J JUNCTIONs. Bioinformatics 20 Suppl 1, 1379-385 (2004).
29. Carlson, C. S. et al. Using synthetic templates to design an unbiased multiplex PCR assay. Nat Commun 4, 2680 (2013).
30. Storey, J. D. & Tibshirani, R. Statistical significance for genomewide studies. Proc Natl Acad Sci USA 100, 9440-9445 (2003).

1.-24. (canceled)

25. A method for predicting a human leukocyte antigen (HLA) allele status of a subject, comprising:
(a) performing high throughput sequencing of genomic DNA obtained from a sample comprising T cells from a subject of unknown HLA allele status to determine a T-cell receptor (TCR) profile comprising unique TCR rearranged DNA sequences;
(b) comparing the TCR profile of the subject with a set of previously identified TCR profiles in a database, wherein each of the previously identified TCR profiles comprises TCR rearranged DNA sequences statistically significantly associated with a known HLA allele status for a plurality of subjects;
(c) generating a score for the subject, wherein the score is a weighted function based on the unique TCR rearranged DNA sequences and/or abundance thereof in the profile of the subject that match the TCR rearranged DNA sequences of the previously identified TCR profiles in the database;
(d) inputting the score from (c) into an algorithm, wherein the algorithm compares the score of the subject and the HLA allele status from the plurality of subjects of known HLA allele status;
(e) determining an estimated probability of the HLA allele status of the subject as an output of the algorithm; and
(f) predicting the HLA allele status of the subject based on the estimated probability determined at step (e).

26. The method of claim 25, wherein the database classifies the plurality of subjects based on (i) the known HLA allele status of the subject and (ii) a presence or absence in the subject's immune receptor profile of a feature comprising a unique TCR rearranged DNA sequence.

27. The method of claim 25, wherein generating the score comprises determining a p-value using a Fisher exact two-tailed test.

28. The method of claim 27, further comprising determining a cutoff p-value for identifying the set of features that are significantly associated with an HLA allele status, wherein the cutoff p-value is less than or equal to 1*10⁻⁴.

29. The method of claim 25, wherein the algorithm comprises a logistic regression model.

30. The method of claim 29, wherein the logistic regression model performs a leave-one out cross validation method for at least one round.

31. The method of claim 25, wherein said HLA allele status is of an HLA-A2 allele or an HLA-24 allele.


SEQUENCE LISTING (163 sequences)

SEQ ID NO: 1 - protein, 13 aa, Homo sapiens
CASSGQGAYEQYF

SEQ ID NO: 2 - protein, 15 aa, Homo sapiens
CASSPDRVGQETQYF

SEQ ID NO: 3 - protein, 15 aa, Homo sapiens
CASSIGPLEHNEQFF

SEQ ID NO: 4 - protein, 13 aa, Homo sapiens
CASSIEGNQPQHF

SEQ ID NO: 5 - protein, 15 aa, Homo sapiens
CASSLVAGGRETQYF

SEQ ID NO: 6 - protein, 13 aa, Homo sapiens
CASSLEAEYEQYF

SEQ ID NO: 7 - protein, 11 aa, Homo sapiens
CATSDGDEQFF

SEQ ID NO: 8 - protein, 16 aa, Homo sapiens
CASSLAPGATNEKLFF

SEQ ID NO: 9 - protein, 13 aa, Homo sapiens
CASSRGRQETQYF

SEQ ID NO: 10 - protein, 15 aa, Homo sapiens
CASSAGQGVTYEQYF

SEQ ID NO: 11 - protein, 12 aa, Homo sapiens
CASSLRREKLFF

SEQ ID NO: 12 - protein, 16 aa, Homo sapiens
CASSLIGVSSYNEQFF

SEQ ID NO: 13 - protein, 15 aa, Homo sapiens
CASSFPTSGQETQYF

SEQ ID NO: 14 - protein, 13 aa, Homo sapiens
CASSPQRNTEAFF

SEQ ID NO: 15 - protein, 13 aa, Homo sapiens
CSVRDNFNQPQHF

SEQ ID NO: 16 - protein, 15 aa, Homo sapiens
CATSRDSQGSYGYTF

SEQ ID NO: 17 - protein, 11 aa, Homo sapiens
CASSPGDEQYF

SEQ ID NO: 18 - protein, 15 aa, Homo sapiens
CASSQTGGRNQPQHF

SEQ ID NO: 19 - protein, 14 aa, Homo sapiens
CASSQNRGQETQYF

SEQ ID NO: 20 - protein, 15 aa, Homo sapiens
CASSLVIGGDTEAFF

SEQ ID NO: 21 - protein, 14 aa, Homo sapiens
CASSFHGFNQPQHF

SEQ ID NO: 22 - protein, 15 aa, Homo sapiens
CASSRLAGGTDTQYF

SEQ ID NO: 23 - protein, 15 aa, Homo sapiens
CASSLPSGLTDTQYF

SEQ ID NO: 24 - protein, 15 aa, Homo sapiens
CATSRDTQGSYGYTF

SEQ ID NO: 25 - protein, 11 aa, Homo sapiens
CATSDGDTQYF

SEQ ID NO: 26 - protein, 15 aa, Homo sapiens
CASSLVASGRETQYF

SEQ ID NO: 27 - protein, 14 aa, Homo sapiens
CASSIWGLDTEAFF

SEQ ID NO: 28 - protein, 11 aa, Homo sapiens
CASSPGDEQFF

SEQ ID NO: 29 - protein, 13 aa, Homo sapiens
CASSPSTGTEAFF

SEQ ID NO: 30 - protein, 14 aa, Homo sapiens
CSVEEDEGIYGYTF

SEQ ID NO: 31 - protein, 13 aa, Homo sapiens
CASSEIPNTEAFF

SEQ ID NO: 32 - protein, 16 aa, Homo sapiens
CASSQVPGQGDNEQFF

SEQ ID NO: 33 - protein, 14 aa, Homo sapiens
CASSPAGLNTEAFF

SEQ ID NO: 34 - protein, 13 aa, Homo sapiens
CASSLGLKGTQYF

SEQ ID NO: 35 - protein, 13 aa, Homo sapiens
CASSGDRLYEQYF

SEQ ID NO: 36 - protein, 13 aa, Homo sapiens
CSVRDNYNQPQHF

SEQ ID NO: 37 - protein, 15 aa, Homo sapiens
CASSYGGLGSYEQYF

SEQ ID NO: 38 - protein, 14 aa, Homo sapiens
CASNRDRGRYEQYF

SEQ ID NO: 39 - protein, 13 aa, Homo sapiens
CASMGGASYEQYF

SEQ ID NO: 40 - protein, 15 aa, Homo sapiens
CASSLGVGPYNEQFF

SEQ ID NO: 41 - protein, 14 aa, Homo sapiens
CASSLGGAGDTQYF

SEQ ID NO: 42 - protein, 14 aa, Homo sapiens
CATSRGTVSYEQYF

SEQ ID NO: 43 - protein, 11 aa, Homo sapiens
CATSDGETQYF

SEQ ID NO: 44 - protein, 15 aa, Homo sapiens
CASSEARGGVEKLFF

SEQ ID NO: 45 - protein, 14 aa, Homo sapiens
CASSLNRGQETQYF

SEQ ID NO: 46 - protein, 13 aa, Homo sapiens
CSVRDNHNQPQHF

SEQ ID NO: 47 - protein, 15 aa, Homo sapiens
CASSESGHRNQPQHF

SEQ ID NO: 48 - protein, 15 aa, Homo sapiens
CSASPGQGASYGYTF

SEQ ID NO: 49 - protein, 12 aa, Homo sapiens
CASSEARTRAFF

SEQ ID NO: 50 - protein, 12 aa, Homo sapiens
CASRPTGYEQYF

SEQ ID NO: 51 - protein, 14 aa, Homo sapiens
CASSVTGGTDTQYF

SEQ ID NO: 52 - protein, 15 aa, Homo sapiens
CASSRLAASTDTQYF

SEQ ID NO: 53 - protein, 15 aa, Homo sapiens
CATSDSVTNTGELFF

SEQ ID NO: 54 - protein, 15 aa, Homo sapiens
CASSRNRESNQPQHF

SEQ ID NO: 55 - protein, 13 aa, Homo sapiens
CASSAQGAYEQYF

SEQ ID NO: 56 - protein, 15 aa, Homo sapiens
CASSIQGYSNQPQHF

SEQ ID NO: 57 - protein, 15 aa, Homo sapiens
CASSYNPYSNQPQHF

SEQ ID NO: 58 - protein, 17 aa, Homo sapiens
CASSLGHRDPNTGELFF

SEQ ID NO: 59 - protein, 13 aa, Homo sapiens
CASSTTGGDGYTF

SEQ ID NO: 60 - protein, 15 aa, Homo sapiens
CASSVLAGPTDTQYF

SEQ ID NO: 61 - protein, 12 aa, Homo sapiens
CASSYRQETQYF

SEQ ID NO: 62 - protein, 13 aa, Homo sapiens
CASSSGQVYGYTF

SEQ ID NO: 63 - protein, 12 aa, Homo sapiens
CASGRDTYEQYF

SEQ ID NO: 64 - protein, 16 aa, Homo sapiens
CATSDSRTGGQETQYF

SEQ ID NO: 65 - protein, 16 aa, Homo sapiens
CASSSPGRSGANVLTF

SEQ ID NO: 66 - protein, 12 aa, Homo sapiens
CASSYGGEGYTF

SEQ ID NO: 67 - protein, 14 aa, Homo sapiens
CASSLAGVDYEQYF

SEQ ID NO: 68 - protein, 13 aa, Homo sapiens
CASSLQGADTQYF

SEQ ID NO: 69 - protein, 13 aa, Homo sapiens
CASSLEAENEQFF

SEQ ID NO: 70 - protein, 16 aa, Homo sapiens
CASSEAPSTSTDTQYF

SEQ ID NO: 71 - protein, 13 aa, Homo sapiens
CASSLEGQQPQHF

SEQ ID NO: 72 - protein, 16 aa, Homo sapiens
CASSLGHRDSSYEQYF

SEQ ID NO: 73 - protein, 13 aa, Homo sapiens
CASSPSRNTEAFF

SEQ ID NO: 74 - protein, 13 aa, Homo sapiens
CSALGHSNQPQHF

SEQ ID NO: 75 - protein, 14 aa, Homo sapiens
CASSHRDRNYEQYF

SEQ ID NO: 76 - protein, 15 aa, Homo sapiens
CASSPPGQGSDTQYF

SEQ ID NO: 77 - protein, 15 aa, Homo sapiens
CASSLQGYSNQPQHF

SEQ ID NO: 78 - protein, 16 aa, Homo sapiens
CASSYVRTGGNYGYTF

SEQ ID NO: 79 - protein, 13 aa, Homo sapiens
CASSRDRNYGYTF

SEQ ID NO: 80 - protein, 15 aa, Homo sapiens
CASSTGTSGSYEQYF

SEQ ID NO: 81 - protein, 14 aa, Homo sapiens
CASRSDSGANVLTF

SEQ ID NO: 82 - protein, 13 aa, Homo sapiens
CATSRVAGETQYF

SEQ ID NO: 83 - protein, 13 aa, Homo sapiens
CASSEEGIQPQHF

SEQ ID NO: 84 - protein, 14 aa, Homo sapiens
CASSLGGPGDTQYF

SEQ ID NO: 85 - protein, 15 aa, Homo sapiens
CASSLVAAGRETQYF

SEQ ID NO: 86 - protein, 14 aa, Homo sapiens
CASRGQGWDEKLFF

SEQ ID NO: 87 - protein, 14 aa, Homo sapiens
CASSLEGQGFGYTF

SEQ ID NO: 88 - protein, 14 aa, Homo sapiens
CASRDWDYTDTQYF

SEQ ID NO: 89 - protein, 17 aa, Homo sapiens
CASSRSGLAGNTGELFF

SEQ ID NO: 90 - protein, 16 aa, Homo sapiens
CASSPGQEAGANVLTF

SEQ ID NO: 91 - protein, 14 aa, Homo sapiens
CASSLGDRPDTQYF

SEQ ID NO: 92 - protein, 13 aa, Homo sapiens
CASSFPGGETQYF

SEQ ID NO: 93 - protein, 12 aa, Homo sapiens
CASSLETYGYTF

SEQ ID NO: 94 - protein, 14 aa, Homo sapiens
CASSSGQVQETQYF

SEQ ID NO: 95 - protein, 12 aa, Homo sapiens
CASSFDNYGYTF

SEQ ID NO: 96 - protein, 13 aa, Homo sapiens
CASSEGARQPQHF

SEQ ID NO: 97 - protein, 15 aa, Homo sapiens
CASSLTGGRNQPQHF

SEQ ID NO: 98 - protein, 13 aa, Homo sapiens
CASSLLWDQPQHF

SEQ ID NO: 99 - protein, 16 aa, Homo sapiens
CASSLFGTGGNTEAFF

SEQ ID NO: 100 - protein, 12 aa, Homo sapiens
CASSISAGEAFF

SEQ ID NO: 101 - protein, 15 aa, Homo sapiens
CASSPPSGLTDTQYF

SEQ ID NO: 102 - protein, 12 aa, Homo sapiens
CASSPLSDTQYF

SEQ ID NO: 103 - protein, 13 aa, Homo sapiens
CASSSRGTGELFF

SEQ ID NO: 104 - protein, 12 aa, Homo sapiens
CASSYAGDGYTF

SEQ ID NO: 105 - protein, 14 aa, Homo sapiens
CASSDRGNTGELFF

SEQ ID NO: 106 - protein, 11 aa, Homo sapiens
CASSPGGTQYF

SEQ ID NO: 107 - protein, 15 aa, Homo sapiens
CASSLGDRAYNEQFF

SEQ ID NO: 108 - protein, 15 aa, Homo sapiens
CASSLRGSSYNEQFF

SEQ ID NO: 109 - protein, 14 aa, Homo sapiens
CASSLTASSYEQYF

SEQ ID NO: 110 - protein, 10 aa, Homo sapiens
CSASDHEQYF

SEQ ID NO: 111 - protein, 14 aa, Homo sapiens
CASSQGRHTDTQYF

SEQ ID NO: 112 - protein, 15 aa, Homo sapiens
CASSRPGQGNTEAFF

SEQ ID NO: 113 - protein, 12 aa, Homo sapiens
CASSLVGDGYTF

SEQ ID NO: 114 - protein, 14 aa, Homo sapiens
CASSLGAGNQPQHF

SEQ ID NO: 115 - protein, 13 aa, Homo sapiens
CASSLTDTGELFF

SEQ ID NO: 116 - protein, 16 aa, Homo sapiens
CASSLTGGNSGNTIYF

SEQ ID NO: 117 - protein, 13 aa, Homo sapiens
CAWRGTGNSPLHF

SEQ ID NO: 118 - protein, 12 aa, Homo sapiens
CASASANYGYTF

SEQ ID NO: 119 - protein, 14 aa, Homo sapiens
CASSLQAGANEQFF

SEQ ID NO: 120 - protein, 14 aa, Homo sapiens
CASSEEAGGSGYTF

SEQ ID NO: 121 - protein, 12 aa, Homo sapiens
CASRTGESGYTF

SEQ ID NO: 122 - protein, 11 aa, Homo sapiens
CASSGLNEQFF

SEQ ID NO: 123 - protein, 14 aa, Homo sapiens
CASSWDRDNSPLHF

SEQ ID NO: 124 - protein, 14 aa, Homo sapiens
CASSIRTNYYGYTF

SEQ ID NO: 125 - protein, 10 aa, Homo sapiens
CSARSPEAFF

SEQ ID NO: 126 - protein, 15 aa, Homo sapiens
CASSRGTGATDTQYF

SEQ ID NO: 127 - protein, 14 aa, Homo sapiens
CASSPRVSNQPQHF

SEQ ID NO: 128 - protein, 15 aa, Homo sapiens
CAISESQDRGHEQYF

SEQ ID NO: 129 - protein, 14 aa, Homo sapiens
CASSLGRGYEKLFF

SEQ ID NO: 130 - protein, 13 aa, Homo sapiens
CSVEVRGTDTQYF

SEQ ID NO: 131 - protein, 13 aa, Homo sapiens
CASRGQGAGELFF

SEQ ID NO: 132 - protein, 14 aa, Homo sapiens
CATSREGSGYEQYF

SEQ ID NO: 133 - protein, 12 aa, Homo sapiens
CASSLGWTEAFF

SEQ ID NO: 134 - protein, 15 aa, Homo sapiens
CASSLGSSSYNEQFF

SEQ ID NO: 135 - protein, 13 aa, Homo sapiens
CASSSAGADTQYF

SEQ ID NO: 136 - protein, 13 aa, Homo sapiens
CASSERKNYGYTF

SEQ ID NO: 137 - protein, 15 aa, Homo sapiens
CASRDRDRVNTEAFF

SEQ ID NO: 138 - protein, 11 aa, Homo sapiens
CASSRVGEQFF

SEQ ID NO: 139 - protein, 13 aa, Homo sapiens
CASSPRWQETQYF

SEQ ID NO: 140 - protein, 11 aa, Homo sapiens
CASTPGDTIYF

SEQ ID NO: 141 - protein, 14 aa, Homo sapiens
CASSENGGNQPQHF

SEQ ID NO: 142 - protein, 12 aa, Homo sapiens
CASSYPGETQYF

SEQ ID NO: 143 - DNA, 39 bp, Homo sapiens
  source 1..39: mol_type = genomic DNA
tgtgccagcagtgggggggcaaacaccgggcagctctac

SEQ ID NO: 144 - DNA, 12 bp, Homo sapiens
  source 1..12: mol_type = genomic DNA
agtgggggggca

SEQ ID NO: 145 - DNA, 39 bp, Homo sapiens
  source 1..39: mol_type = genomic DNA
tgtgccagcagtgggggggcgaacaccgggcagctctac

SEQ ID NO: 146 - DNA, 12 bp, Homo sapiens
  source 1..12: mol_type = genomic DNA
agtgggggggcg

SEQ ID NO: 147 - DNA, 39 bp, Homo sapiens
  source 1..39: mol_type = genomic DNA
tgtgccagcagcgggggggcaaacaccgggcagctctac

SEQ ID NO: 148 - DNA, 12 bp, Homo sapiens
  source 1..12: mol_type = genomic DNA
agcgggggggca

SEQ ID NO: 149 - protein, 13 aa, Homo sapiens
CASSGGANTGQLY

SEQ ID NO: 150 - protein, 14 aa, Homo sapiens
CASSLEAGAQPQHF

SEQ ID NO: 151 - protein, 13 aa, Homo sapiens
CASSIRSGVEQYF

SEQ ID NO: 152 - protein, 14 aa, Homo sapiens
CSARDRGIGNTIYF

SEQ ID NO: 153 - protein, 13 aa, Homo sapiens
CASSQSPGGTQYF

SEQ ID NO: 154 - protein, 12 aa, Homo sapiens
CASSPETGEAFF

SEQ ID NO: 155 - protein, 13 aa, Homo sapiens
CASSLRSTGELFF

SEQ ID NO: 156 - protein, 14 aa, Homo sapiens
CASSENSPHQPQHF

SEQ ID NO: 157 - protein, 12 aa, Homo sapiens
CASSQEAGPQHF

SEQ ID NO: 158 - protein, 14 aa, Homo sapiens
CASSIRDRGQPQHF

SEQ ID NO: 159 - protein, 13 aa, Homo sapiens
CASSYQGGNEQFF

SEQ ID NO: 160 - protein, 12 aa, Homo sapiens
CASSGGANVLTF

SEQ ID NO: 161 - protein, 12 aa, Homo sapiens
CSVEDLGYGYTF

SEQ ID NO: 162 - protein, 15 aa, Homo sapiens
CASRRGGSSYNEQFF

SEQ ID NO: 163 - protein, 11 aa, Homo sapiens
CASRSGTEAFF